(19) United States
(12) Patent Application Publication
(10) Pub. No.: US 2013/0006040 A1
(43) Pub. Date: Jan. 3, 2013

(54) MASSAGE APPARATUS USING MAGNET IS THE COSMETIC CONTAINER ADHERED TO LOWER PART

(76) Inventor: Yong-Sik Lee, Seoul (KR)

(21) Appl. No.: 13/634,604

(22) PCT Filed: Mar. 17, 2011
(86) PCT No.: PCT/KR2011/001853

§ 371 Date: Sep. 13, 2012

(30) Foreign Application Priority Data
Mar. 18, 2010 (KR) 10-2010-0024172

(51) Int. Cl. A61N 2/08; A61N 1/30

(52) U.S. Cl. 600/15

(57) ABSTRACT

[Summary] The invention relates to the cosmetic container comprising the masseur machine in which the carrying having the simple structure where the massage apparatus which puts the magnetic material and in which the magnet is adhered to the receiving part lower part bottom surface of the cosmetic container is combined is convenient and more specifically, the attaching/detaching is easy on the massage apparatus using the magnet as the invention relating to the cosmetic container adhered to the lower part.

Characteristic the massage apparatus using magnet of this kind of invention as to the cosmetic container adhered to lower part, has the lid part who enables to detach and attach is combined on the top of receiving part having the accommodation space of the massage apparatus part: material which enables to detach and attach is installed at the lower part of the receiving part top is open the lower part is shut tightly: receiving part as the configuration.

### TECHNICAL FIELD

The invention relates to the cosmetic container comprising the secondary masseur machine in which the cosmetics is effectively introduced to the internal.

### BACKGROUND THE DESCRIPTION

Generally, in the lower part of the keratin layer of the skin of skin, the firewall consisting of the warm protein is formed. The material including the cosmetics applied to skin of skin skin and Aurantii nobilis Pericarpium are separated of skin, the nutriments, the medicine etc. is unable to be deeply infiltrated with this protein firewall to the Aurantii nobilis Pericarpium. Various skin care assistant device etc using the iontophoresis using galvanic ionic current (the direct current in which it has the positive electric current (+) and negative current (−)) this material is deeply infiltrated to the Aurantii nobilis Pericarpium, and cold and warmth masseur machine etc using the magnetic field of the magnet, and the optical irradiation, the ultrasound massage, the far infrared ray, the low frequency of the laser or LED and thermoelectric element are developed.

The cosmetic container and attaching/detaching were facilitated and the carrying was this skin care assistant device convenient and it was together used according to the property of the aqueous solution of the cosmetics.

In the assistant device using the magnet, in other words, the magnetic field, the contained Fe component is touched with the hemoglobin of the blood to N pole or the south pole and the function of the hemoglobin is activated and the oxygen transport, and the feeding and waste emission smooth and the skin trouble disappears with the blood row effect and the skin gets clean.

The galvanic ionic current may be referred to the instrument authorizing the ionic current in skin and the iontophoresis skin care assistant device which uses induces the movement of the skin ion. If the ionic current is authorized in skin because of being the bar using the skin care assistant device using this galvanic ionic current ions in which the galvanic ionic current exists in skin in and out are moved and the ion energy is generated. It easily relaxes the nerve of skin and this ion energy deeply penetrates the material to the Aurantii nobilis Pericarpium.

In the assistant device using ultrasound is the vibration seconds 100 bay, the high speed massage occurs in the deep with skin tissue place due to the high speed vibration of 180. The effect that the large help is in the cosmetic effects and health is offered concerning to the flow of the lymph of face be improved with the minute high speed massage and lymph is soaked to the difficult cell gap interval that blood penetrates and it functions with metabolism be smooth of the flow.

The laser called light illuminating apparatus or the assistant device using LED gives the magnetic pole to skin and the laser called light illuminating apparatus or the assistant device using LED accelerates the circulation of blood. Metabolism the skin disease is highly prevented with the laser irradiator as well as the box. The magnetic pole is given to the affected part of the patient in which the patient has the skin disease and the skin disease can be treated.

As to the assistant device, using cold and warmth massage using the thermoelectric element the COOL function decreases the help of the pore tightening and skin firmness and the HOT function gives the firewall relax of the skin and help of the circulation of the blood.

There is a problem that it is difficult and the carrying is inconvenient that it is known to have this for massage assistant devices but the apparatus is big and the apparatus is complicated and it is the high price and general persons easily buy.

### DETAILS OF THE INVENTION

The invention provides the cosmetic container having the feature including the carrying is the simple masseur machine the attaching/detaching is possible through the simple structure at the lower part of the receiving part of the cosmetic container.

It characteristics, the massage apparatus part attached the magnet of the present invention with the magnetic material installed at the bottom surface which lower part is sealed the receiving part while it characteristics, constituting the lid part who can be detached and attached, is combined on the top of receiving part having the accommodation space of the massage apparatus part: material which can be detached and attached, the cosmetics container in which the massage apparatus which uses is attached to the lower part is installed at the lower part of the receiving part the top is opened the lower part is shut tightly: receiving part to configuration that the ground is combined with the work nest in which the magnet is built in and the separator which is to insulator.

Here, the cosmetics container in which the massage apparatus using magnet is attached to the lower part of the present invention is equipped with the ground electrode who the massage apparatus part combined with the lower part of the receiving part provides the positive electric current (+) or the negative current (−) from battery, and the working electrode part supplying to the human body which the negative current (−) of the ground electrode and the other polarity or the positive electric current (+) is provided from battery in order to be insulated with the ground electrode while the electrode separation part is installed and touched.

In the meantime, the cosmetic container adhered to the massage apparatus used is the lower part the magnet of the present invention can further include the working electrode and ground electrode the respective negative current (−) or the circuit board which supplies the positive electric current (+) and the display lamp which is installed in the circuit board and indicates the decided operation of the working electrode it has the battery and electronic circuit it is installed in the electrode separation embolium opened in the ground electrode insulates the ground electrode and working electrode liver it is installed between the power switch: ground electrode which switches the current supply to the working electrode part in the electronic circuit it is installed in the outer circumference of the electrode separation part or the ground electrode and working electrode.

In the meantime, the outside was made of the transparent material so that the light of the display lamp was transmitted to the outside or the electrode separation part of the present invention lit the display lamp through the through-hole of the uncertain electrode separation part. Moreover, it has with the number drawing is turned on with bay when connecting the human detection sensor to the display lamp and contacting to the skin in which the user catches by hand the ground electrode and which the user calls.

In the meantime, while it can be detached and attached, the lower part of the working electrode part is combined on the top of the electrode separation part of the present invention, the penetration hole is formed.

In the meantime, the electronic circuit of the present invention is equipped with the electric current supply which generates the negative current (−) and positive electric current (+) it is provided with the power source from the battery and the polar conversion part who mutually converts the polarity of the positive electric current (+) and respective applied negative current (−) to the working electrode and ground electrode.

In the meantime, it is installed in the outer circumference of the bottom surface of the negative current generated in the voltaic occurring part and the amplifier: controller: sanctioning the amplified negative current (−) as described above and positive electric current (+) to the respective working electrode and ground electrode and ground electrode or the electrode separation part and amplifies the positive electric current the electronic circuit of the present invention more includes the polarity changeover switch in which user can control the polar conversion part.

In the meantime, in order to the power supply unit of the present invention battery to the secondary battery and it charges the charging apparatus which is combined with the massage apparatus part attached at the lower part of the cosmetics container independenting can be established.

In the meantime, in the lower part of massage apparatus part and receiving part of the present invention, it is able to detach and attach in each other the magnetic material and magnet are installed. Moreover, it can be installed in the corresponding coupling structure in the coupling structure of the lock shape of the concavo-convex or the female screw and male screw.

In the meantime, the massage apparatus using the magnet is the cosmetic container adhered to the lower part of the present invention is equipped with the power operation switch which the illuminating unit irradiating light is installed at the inside while the massage apparatus part combining with the lower part of the receiving part the penetration hole is formed in the work nest and is provided with the current from the electronic circuit and switches through the led or the laser unit to the work nest in the connecting part or the electrode separation large outer circumference while irradiating light.

In the meantime, the massage apparatus using the magnet is the cosmetic container adhered to the lower part of the present invention is equipped with the power operation switch which the massage apparatus part combining with the lower part of the receiving part switches in the ground electrode or the electrode separation large outer circumference while being provided with the ultra-audible frequency from the electronic circuit and generating ultrasound through the piezoelectric vibrator of the working electrode part.

In the meantime, the cosmetics container in which the massage apparatus using magnet is attached to the lower part of the present invention is equipped with the working electrode part in which the flight board is the massage apparatus part combined with the lower part of the receiving part provides cold and warmth frequency from the electronic circuit and ground electrode or the power operation switch switching in the electrode separation large outer circumference the coldness or the heat is occurred through cold and warmth heat burnout person.

In the meantime, the massager of the present invention attached Cosmetic containers cosmetics containers and detachable massaging of the skin contact surface electrode grounded in plasma, plasma is generated by a cold or hot facial lymphatic flow nicely and inflammation and treatment effects.

### BRIEF EXPLANATION OF THE DRAWING

FIG. 1 is a perspective view showing the configuration of the cosmetics container in which the massage apparatus using the magnet of the present invention is attached to the lower part.

FIG. 2 is an isolation showing the massage apparatus using the magnet according to the embodiment 1 of the present invention is the configuration of the cosmetic container adhered to the lower part.

FIG. 3 is a side sectional view showing the electromagnet massage apparatus using the magnet according to the embodiment 1 of the present invention is the configuration of the cosmetic container adhered to the lower part.

FIG. 4 is an isolation showing the ionization massage apparatus using the magnet according to the embodiment 2 of the present invention is the configuration of the cosmetic container adhered to the lower part.

FIG. 5 is a side sectional view showing the ion massage apparatus using the magnet according to the embodiment 2 of the present invention is the configuration of the cosmetic container adhered to the lower part.

The magnet according to the embodiment 3 of the FIG. 6 silver the invention may be referred to the isolation showing the configuration of the cosmetic container adhered to the iontophoresis device used is the lower part.

FIG. 7 is a side sectional view showing the iontophoresis device using the magnet according to the embodiment 3 of the present invention is the configuration of the cosmetic container adhered to the lower part.

FIG. 8 is isolation showing the configuration of the cosmetics container in which the laser irradiation apparatus using the magnet according to the working example 4 of the present invention is attached to the lower part.

FIG. 9 is a side sectional view showing the light irradiation apparatus using the magnet according to the embodiment 4 of the present invention is the configuration of the cosmetic container adhered to the lower part.

FIG. 10 is isolation showing the configuration of the cosmetics container in which the vibration massage apparatus using the magnet according to the working example 5 of the present invention is attached to the lower part.

FIG. 11 is a side sectional view showing the ultrasonic massage device using the magnet according to the embodiment 5 of the present invention is the configuration of the cosmetic container adhered to the lower part.

FIG. 12 is an isolation showing cold and warmth massage apparatus using the magnet according to the embodiment 6 of the present invention is the configuration of the cosmetic container adhered to the lower part.

FIG. 13 is a side sectional view showing cold and warmth massage apparatus using the magnet according to the embodiment 6 of the present invention is the configuration of the cosmetic container adhered to the lower part.

FIG. 14 is a side sectional view showing the display in which the cosmetic container adhered to the lower part of the magnet massage apparatus using the magnet according to the embodiment 1 of the present invention is combined.

FIG. 15 is a side sectional view showing the display in which the cosmetic container adhered to the lower part of the electromagnet massage apparatus using the magnet according to the embodiment 1 of the present invention is combined.

FIG. 16 is a side sectional view showing the display in which the cosmetic container adhered to the lower part of the ionization massage apparatus using the magnet according to the embodiment 2 of the present invention is combined.

FIG. 17 is a side sectional view showing the iontophoresis massage apparatus using the magnet according to the embodiment 2 of the present invention is the cosmetic container adhered to the lower part, and, the other combined display.

FIG. 18 is a side sectional view showing the display in which the cosmetics container in which the laser irradiation apparatus using the magnet according to the working example 4 of the present invention is attached to the lower part is combined.

FIG. 19 is a side sectional view showing the display in which the cosmetic container adhered to the lower part of the light massage apparatus using the magnet according to the embodiment 4 of the present invention is combined.

FIG. 20 is a side sectional view showing the display in which the cosmetic container adhered to the lower part of the vibration massage apparatus using the magnet according to the embodiment 5 of the present invention is combined.

FIG. 21 is a side sectional view showing the display in which the cosmetic container adhered to the lower part of the ultrasonic massage device using the magnet according to the embodiment 5 of the present invention is combined.

FIG. 22 is a side sectional view showing the display in which the cosmetics container in which cold and warmth massage apparatus using the magnet according to the working example 6 of the present invention are attached to the lower part is combined.

FIG. 23 is a side sectional view showing the combined display of the battery charger of the cosmetics container in which the massage apparatus using the magnet of the present invention is attached to the lower part.

### FOR CARRYING OUT THE INVENTION THE BEST FORM OF

The detailed content for the attached operation of the present invention is hereinafter illustrated.

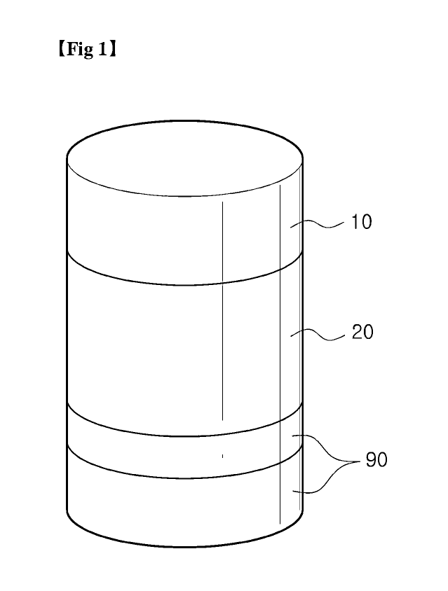

FIG. 1 is a perspective view of the cosmetics container in which the massage apparatus using magnet is attached to the lower part of the invention. Receiving part (10) having accommodation space: thing having the massage apparatus part (90) it can be detached and attached, the magnet is installed and the skin is massaged in the lower part of the receiving part and the lid part (20): which can be detached and attached, is combined in the opened top of the receiving part of material which magnetic material (11) is attached to the bottom surface in the cosmetics container (1), the top is opened and the lower part is shut tightly are constituted with feature.

### BEST MODE FOR CARRYING OUT THE INVENTION

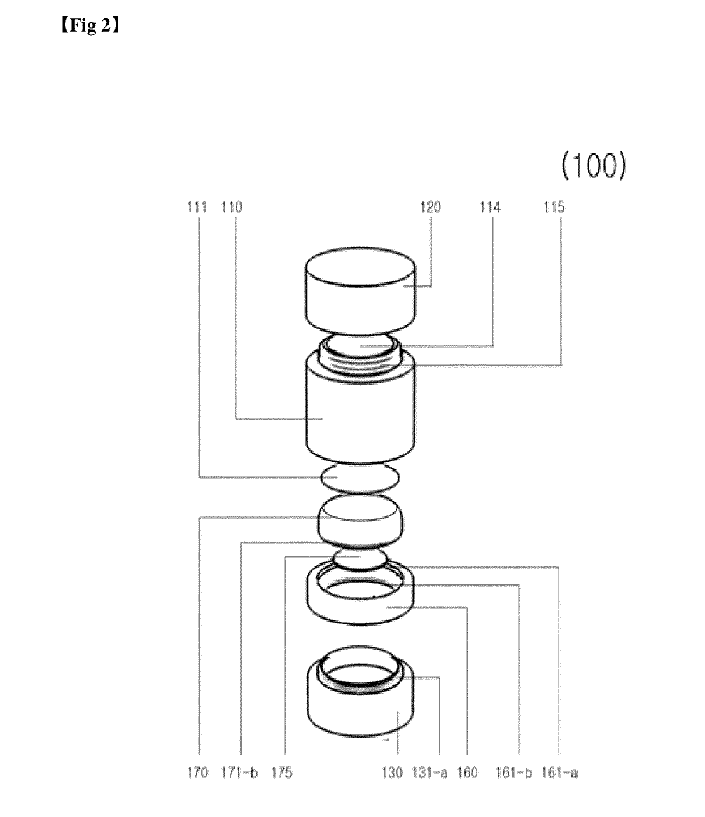

FIG. 2 is a perspective view showing the massage apparatus using the magnet according to the embodiment 1 of the present invention is the configuration of the cosmetic container adhered to the lower part. FIG. 2 is a side sectional view showing the electromagnet massage apparatus using the magnet according to the embodiment 1 of the present invention is the configuration of the cosmetic container adhered to the lower part.

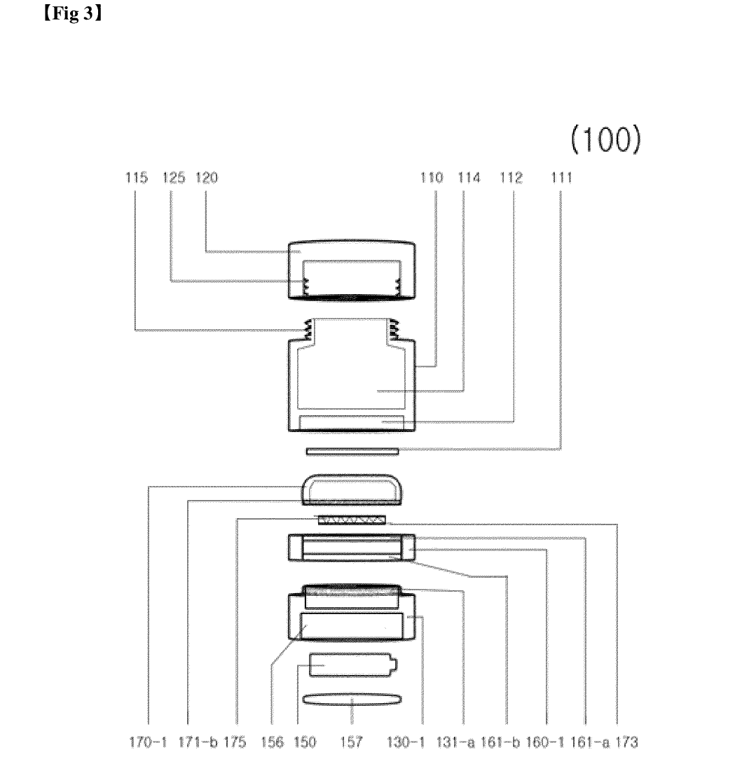

As shown in FIGS. 2 and 3. And the cosmetics container (100) in which the massage apparatus using magnet according to the working example 1 is attached to the lower part of the present invention is equipped with the receiving part (110), the lid part (120) and massage apparatus part (190) etc.

In order to have the accommodation space (114) in which the receiving part (110) is the material of the cosmetic, medicine, nutrient, the powder etc is put the top is opened and the lower part is formed into the shape of the cylinder shut tightly or the various angles cask. In the outer circumference of the receiving part (110) top, the male screw (115) is formed as the regular length so that the female screw (125) of the lower part of the lid part (120) which will be described later be combined.

The magnetic material (111), and, the other magnet is installed so that the magnet (magnet) (175) built in of the cradle groove (112) is formed in the lower part bottom surface the lower part of the receiving part (110) was shut tightly so that the massage apparatus part (190) be settled the massage apparatus part (190) be attached. In the inner circumference of the lid part (120), the female screw (125) in which the female thread is formed as the regular length is formed so that the male screw (115) formed on the top of the receiving part (110) be screwed. Even if configuration is each other opposed, the joint like above mentioned male screw (115) and female screw (125) can have the same function, it self-evidents.

While the massage apparatus part (190) the work nest (170) and the separator (160) which separates the dipolar liver while being comprised of the connecting part (130) are installed in the polygon or the cylinder the magnet (magnet) (175) of S or N pole is adhered to the work nest (170) bottom surface with the lid part (120) top to attach and can detach. And the separator (160) is made of the polygon or the cylindrical shape and the penetration hole (162) is formed in the inside.

The bottom surface of the work nest in which the magnet (magnet) (175) was adhered was distinguished into the south pole and the opposite surface of the magnet (magnet) (175) was distinguished into N pole and N pole was connected to the connecting part as the spring (155) and the direction of the magnetic field flowed from N pole to the south pole. The related has the flow of the magnetic field with—electrode with the flowing direction of the current. According to the property of the product, in order to flow from the south pole to N pole the polarity of the magnet is changed and it can set up.

Moreover, the current is away passed and the magnetic field is produced in the form of the electromagnet (electro-magnet) to humanity and justice and the using the battery the coil (176) of the metal having with the penetration pestle is wound instead of the magnet (magnet) (175) while being switched to the battery can have the magnetic field as the power switch misrepresentation operation with the pulsed. At this time, the sticking shape is installed in the corresponding coupling structure in the cosmetic container and massage apparatus part (190) in the coupling structure of the lock shape of the concavo-convex or the female screw and male screw.

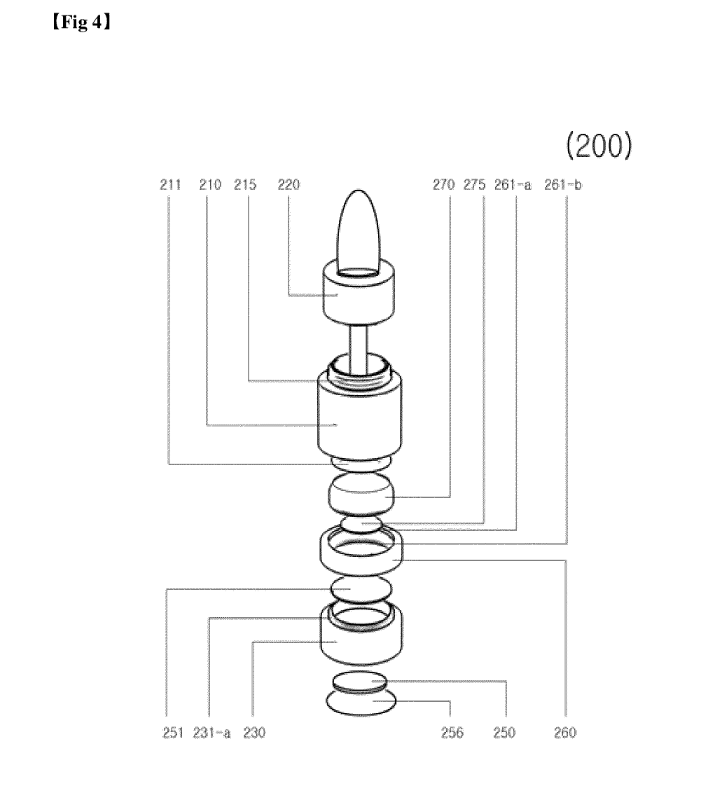

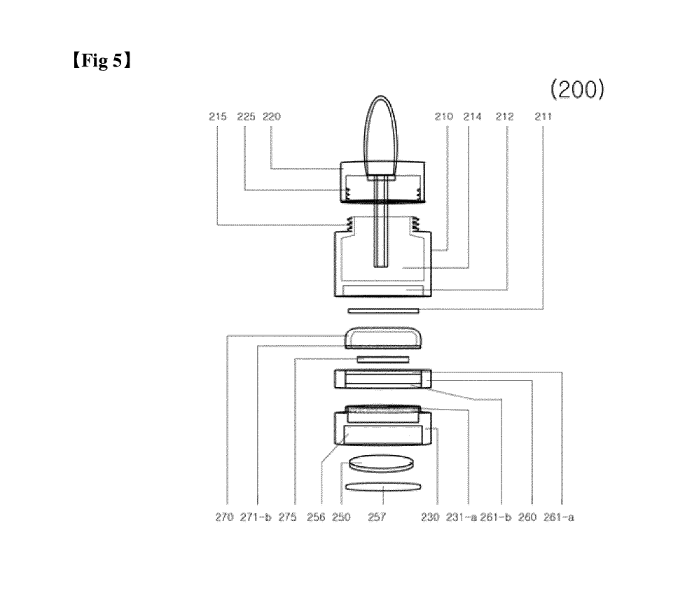

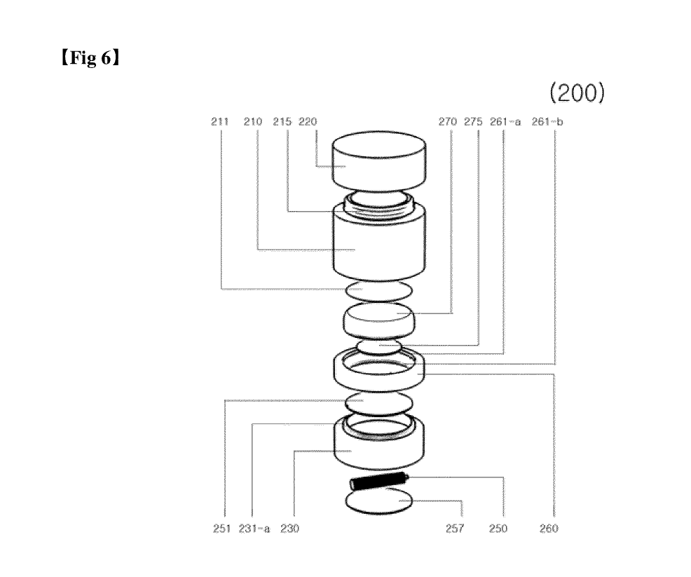

FIG. 4 is isolation showing the configuration of the cosmetics container in which the ionization massage apparatus using the magnet according to the working example 2 of the present invention is attached to the lower part. FIG. 5 is a side sectional view showing the configuration of the cosmetics container in which the ionization massage apparatus using the magnet according to the working example 2 of the present invention is attached to the lower part.

As shown in FIGS. 4 and 5. And the massage apparatus using the magnet according to the embodiment 2 is the cosmetic container (200) adhered to the lower part of the present invention is equipped with the receiving part (210), the lid part (220), and the ground electrode (230), the battery (250), the power switch (240), the electrode separation large (260), the display lamp (280), the working electrode part (270) etc of the massage apparatus part (290).

In order to be made of the shape of the cylinder and the lower part is opened and the lid part (220) is combined with the male screw (215) formed on the top of the receiving part (210) in the inner circumference of the lower part of the lid part (220) the female screw (225) is formed as the regular length.

While being combined at the lower part of the receiving part the massage apparatus part (290) is made of the shape of the cylinder and while being comprised of the working electrode part (270), the electrode separation large (260) and ground electrode (230) the magnet (magnet) (275) for the magnetic material (221) of the receiving part bottom surface and attaching are built-in in the working electrode part (270).

While the switch (240) switching to the power source of the battery (250) is placed while as to the ground electrode (230), the lower part is shut tightly with the shape of cylinder and the top is opened and it is shut tightly and the battery opening/closing device is composed in the lower part bottom surface, while the snuggly wall (non illustration) is formed, joints (261-b, 231-a) which each other cope with are formed in the outer circumference of the upper part of the ground electrode (230) in the lower part of the electrode separation large (260). Even if configuration is each other opposed, joints (261-b, 231-a) can have the same function, it self-evidents. In the meantime, the ground electrode (230) each other has the opposite polarity of the polarity of the working electrode part (270) which will be described later. In the battery (250), the power source is applied and the ion transmission effect can be given as the operation of the power switch (240) in which the lead line (253) is connected to the working electrode part (270) and switched and ground electrode (230). The working electrode part (270) and ground electrode (230) use the synthetic resin coated to metal or the conductive material so that current flow.

It is installed between the ground electrode (230) and working electrode part (270) and the electrode separation large (260) insulates the ground electrode and working electrode liver. This electrode separation large (260) is made of the cylindrical shape and the penetration hole (262) is formed in the inside so that the top of the ground electrode (230) is inserted. And in the inner circumference of the electrode separation large (260), joints (261-a,260-b) having the corresponding coupling structure of the working electrode part (270) which will be described later or the ground electrode (230) are formed. It makes uncertain and the through-hole (281) is formed on the outer circumference and the display lamp (280) can be set up.

Moreover, it is formed into the transparent material and the gradual rise light of the display lamp (280) is transmitted in the lighting of the display lamp (280) which will be described later to the outside of the electrode separation large (260).

It is made of 2 * * * acquaintance nickel cadium capable of 1 * * * acquaintance dry cell, mercury cell, lithium battery, manganese battery or the charge, and the nickel-hydrogen electric cell etc. 2 * * * acquaintance nickel cadium capable of 1 * * * acquaintance dry cell, mercury cell, lithium battery, manganese battery or the charge the battery (250) generates the direct current while being called as the galvanic cell or the Volta cell.

Because of being the bar using the polarity of the current as the negative current (−) or the positive electric current (+) of the battery (250) the ionic current is generated and it can massage with the magnetic field ion. Moreover, in order to charge 2 * * * acquaintance charging battery with electricity the independent battery charger (700) which is combined with the masseuse (290) is installed.

In the meantime, the power switch (240) is connected to the lead line (238) through the perimeter wall through-hole (235) of the ground electrode (230) to the battery (250) or the simple circuit board (251). According to the power switch (240) is the form of the container, it can be installed at the electrode separation large (260).

The working electrode part (270) supplies to the human body which provides the negative current (−) or the positive electric current (+) from the battery (250) while being installed on the top of the electrode separation large (260) and is touched. And the simple circuit (non illustration) connected through the battery (250) is installed so that the display lamp light in the skin-attached of the working electrode part.

The simple circuit connected to transistor, diode and resistance is necessary for the lighting of the display lamp (280) in which as to the simple circuit as described above, the ionization possibles with the power source of the battery (1.5V) but indicating the current flow of the skin. The working electrode part (270) in which the contact unit of the skin becomes and the ground electrode (230) in which the ground for the ionization of the human body becomes use the synthetic resin coated to metal or the conductive material so that current flow.

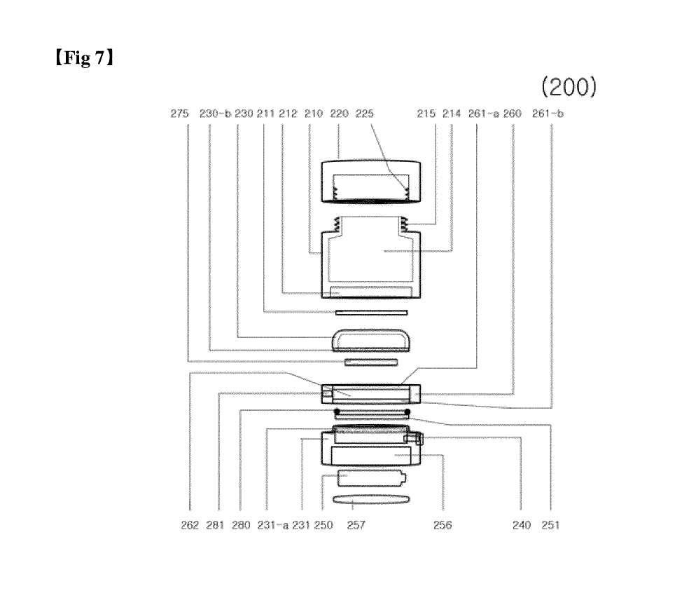

The isolation showing the configuration of the cosmetics container in which the iontophoresis massage apparatus using magnet according to the working example 3 of the FIG. 6 silver the invention is attached to the lower part. FIG. 7 is a side sectional view showing the configuration of the cosmetics container in which the iontophoresis massage apparatus using the magnet according to the working example 3 of the present invention is attached to the lower part.

As shown in the FIG. 6 and FIG. 7. And the massage apparatus using the magnet according to the embodiment 3 is the cosmetic container (200) adhered to the lower part of the present invention is equipped with the receiving part (210), the lid part (220), the ground electrode (230), the battery (250), the circuit board (251), the power switch (240), the display lamp (280), the electrode separation large (260), the working electrode part (270) etc.

The cradle groove (212) is formed in the receiving part (210) lower part bottom surface. And the magnetic material (211) is settled. Moreover, in the inner circumference of the lower part of the lid part (220) which the receiving part top bonds together, in order to be screw-coupled with the male screw (215) formed on the top of the receiving part (210) the female screw (225) is formed as the regular length.

While it is made of the shape of the cylinder and the shape is comprised of the working electrode part (270), the electrode separation large (260) and ground electrode (230) the magnet (magnet) (275) is adhered in the working electrode part (270) bottom surface and the massage apparatus part (290) is installed.

While in the ground electrode (230), the lower part is shut tightly and the top is opened and the circuit board (251) of the electronic circuit and battery (250) is placed, the snuggly wall (non illustration) is formed. In the meantime, the ground electrode (230) each other has the opposite polarity of the polarity of the working electrode part (270) which will be described later.

The battery (250) is fixed in the back side while the circuit board (251) is laid in the ground electrode (230) or the electrode separation large (260). In the back side of the circuit board (251), the lead line (253) which electrically connects the battery (250) and ground electrode (230) is installed. It has the MCU (micro controller unit) electronic circuit (the not illustrated) for generating the ion migration current (the galvanic ionic current) using the power source of the battery (250) in the circuit board (251).

In the meantime, the power switch (240) is connected through the through-hole (235) of the electrode separation large (260) or the ground electrode (230) to the MCU (micro controller unit) electronic circuit (not illustrated) of the circuit board (251). According to this power switch (240) is the operation of the user, the current supply to the ground electrode (230) and working electrode part (270) is switched in the MCU (micro controller unit) electronic circuit (not illustrated).

In the meantime, is side installed through the circuit board (230) the circumference of the electrode separation large (260) and the display lamp (280) indicates the decided operation of the working electrode part (270). The display lamp (280) can comprise so that it is turned on if the human body is contacted with the contact unit (not illustrated) with the human detection sensor (not illustrated) separately installed at the contact unit (not illustrated).

The electronic circuit (not illustrated) comprises the working electrode (negative principle in nature (−) current) the amplifier (not illustrated), amplifying the current generated in the electric current supply (not illustrated), which is provided with the power source from the battery (250) and generates the ion migration current and electric current supply, in other words, the ion migration current and amplified ion migration current as described above, the controller whom it is applied to the ground electrode (amount (+) current), and the MCU (micro controller unit) (not illustrated) etc. And the circuit board (251) of the present invention can further include the working electrode part (270) and the polar conversion part (the not illustrated) which mutually converts the polarity of the flowing current to the ground electrode (230). And in the outer circumference of the back side (in other words, the back side of the ground electrode (230)) of the circuit board (251) or the working electrode part (270), the polarity changeover switch (the not illustrated) in which the user can control the polar conversion part (the not illustrated) can be more prepared.

It is installed between the ground electrode (230) and working electrode part (270) and the electrode separation large (260) each other severs one's connection with the ground electrode and working electrode liver. This electrode separation large (260) is included of the shape of cylindrical and the penetration hole (262) is formed in inside so that the lower part of the ground electrode (230) be inserted. Moreover, in the perimeter wall of the working electrode part (270), the switch (240) which switches the power source while being formed the through-hole (235) is installed. And in the inner circumference of the electrode separation large (260), in order to be combined with the connection part of the working electrode part (270) which will be described later or the ground electrode joints (261-a, 261-b) which each other cope with are formed as the regular length. It is formed into the transparent material and this electrode separation large (260) is transmitted the gradual rise light of the display lamp (280) in the lighting of the display lamp (280) which will be described later to the outside of the electrode separation large (260). Moreover, the electrode separation large (260) is uncertained and the through-hole (281) is formed on the outer circumference and the display lamp (280) can be established

It is installed in the receiving part (210) lower part bottom surface at attach with the magnetic material (211) but the magnet (magnet) (275) can massage with the ionization of the circuit amplified in skin-attached the magnetic field.

It is formed on the top of the main body and it is even so that the area which is contacted with the human body while the material is coated widens the contact unit (not illustrated) is formed.

The main body and circuit board (251) are connected electrically through the lead line (253) installed inside the main body reciprocity.

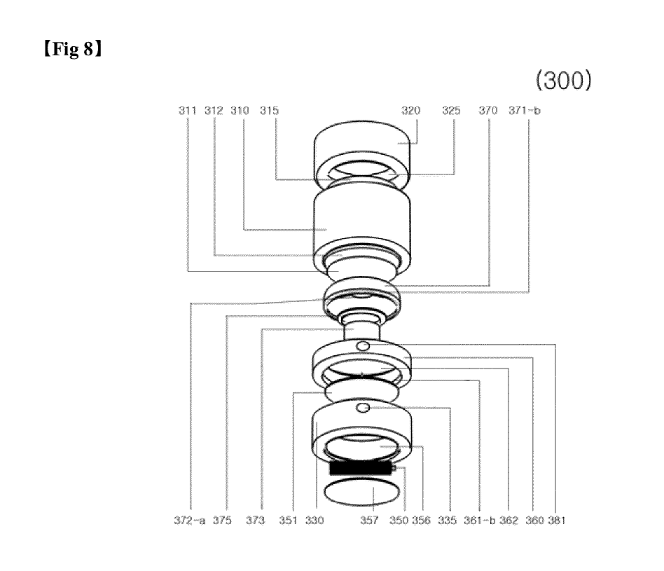

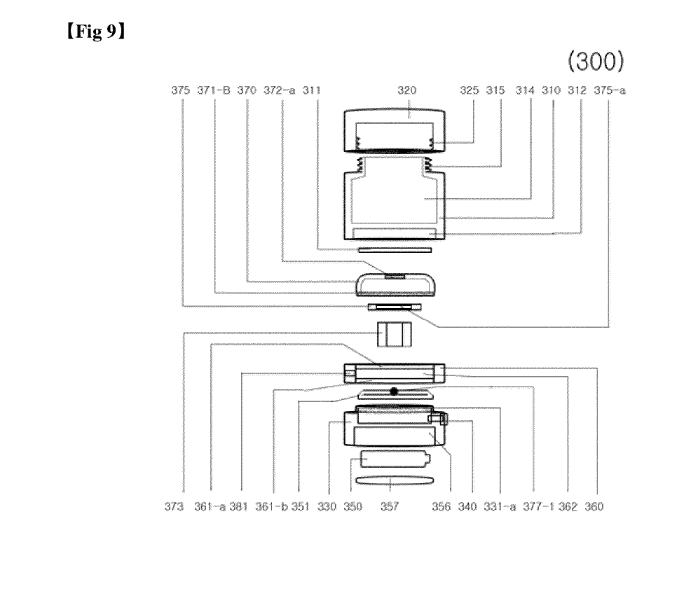

FIG. 8 is an isolation showing the laser irradiation apparatus using the magnet according to the embodiment 4 of the present invention is the configuration of the cosmetic container adhered to the lower part. FIG. 9 is a side sectional view showing the optical irradiation ion massage apparatus using the magnet according to the embodiment 4 of the present invention is the configuration of the cosmetic container adhered to the lower part.

The massage apparatus using the magnet according to the embodiment 4 of the present invention as shown in FIGS. 8 and 9, is the cosmetic container (300) adhered to the lower part can further include the receiving part (310), the lid part (320), the connecting part (330), the ground electrode (330-1), the battery (350), the circuit board (351), the laser-irradiated region (377), the LED illuminating unit (377-1), the power switch (340), the display lamp (380), the separator (360), the electrode separation large (360-1), the work nest (370), the working electrode part (370-1) etc.

In order to have the accommodation space (314) in which FIG. 8 the receiving part (310) is the material of the cosmetics, medicine, nutrient, powder etc is put top is open and the lower part is formed with the shape of the sealed cylinder or the various angles cask.

It is made of the shape of the cylinder and the lower part is shut tightly and the cradle groove (312) is formed and the magnetic material (311) is built in the bottom surface or the receiving part (310) is adhered. Moreover, in the inner circumference of the lower part of the lid part (320), in order to be screw-coupled with the male screw (315) formed on the top of the receiving part (310) the female screw (325) is formed as the regular length.

It is included of the shape of cylinder and the massage apparatus part (390) is composed of the work nest (370) and ground (330). And penetration holes (372-a, 375-a) are formed in the work nest (370) and the magnet (magnet) (375) attached to the work nest (370) bottom surface. The laser-irradiated region (377) which examines laser the guide tube (373) by using the power source in which the guide tube (373) is installed between penetration holes (372-a, 375-a) and provided from the circuit board (351) is established more. According to the laser-irradiated region (377) is distance with the skin of the contact unit (non illustration), in order to examine laser if the human body touches with the distance sensing sensor (non illustration) in the contact unit (non illustration) it can constitute.

Moreover, in order to share with the ionization massage while the working electrode part and the electronic circuit (non illustration) which occurs the ion migration current (galvanic ionic current) by the electrode separation large (360-1) in which the penetration hole (362) is formed is established in inside in order to the ground electrode and the working electrode and ground electrode liver are one's connection with severed each other and using the power source of the battery (350) in the circuit board (351) being mounted the work nest (370) and ground (330) and examining the light it is installed. Here, the optical irradiation explains the light of LED.

The light treatment using LED the laser irradiation helps while improving the blood row helps.

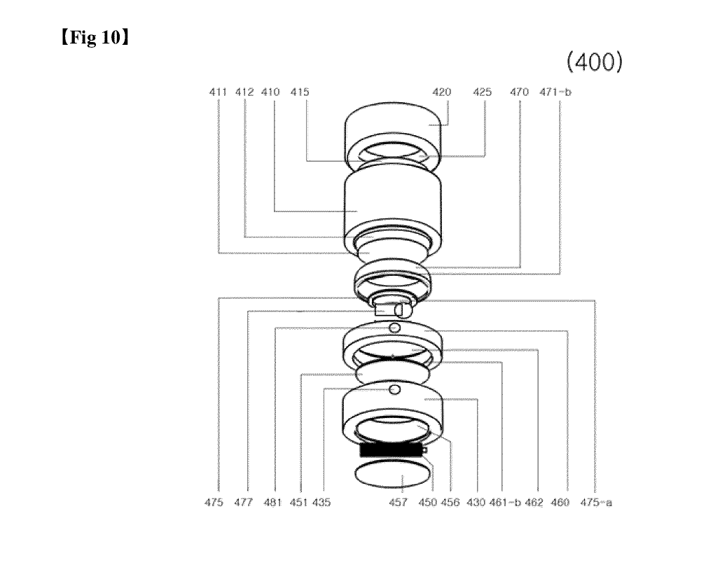

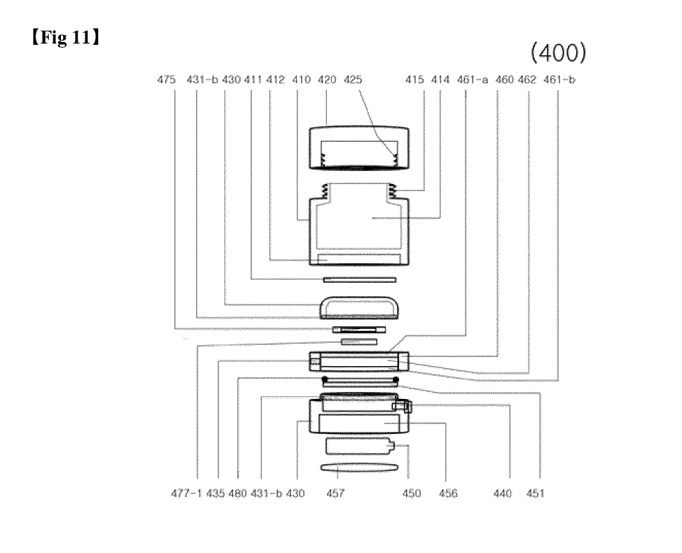

FIG. 10 is an isolation showing the vibration massage apparatus using the magnet according to the embodiment 5 of the present invention is the configuration of the cosmetic container adhered to the lower part. FIG. 11 is a side sectional view showing the ultrasonic massage device using the magnet according to the embodiment 5 of the present invention is the configuration of the cosmetic container adhered to the lower part.

As shown in FIG. 10. And the vibration massage apparatus using the magnet according to the embodiment 5 is the cosmetic container (400) adhered to the lower part of the present invention is equipped with the receiving part (410), the lid part (420) ground electrode (430), the battery (450), the circuit board (451), the power switch (440), the display lamp (480), the electrode separation large (460), the working electrode part (470), the small-sized vibrating motor (477) etc.

The receiving part (410) is made of the shape of the cylinder and top is open and the lower part is shut tightly and the cradle groove (412) is put in the bottom surface and the magnetic material (411) is placed. In the inner circumference of the lower part of the lid part (420), in order to be screw-coupled with the opened male screw (415) formed in upper part which of the receiving part (410) the female screw (425) is formed as the regular length. Even if the configuration is opposed it can have the same function the joint like above mentioned male screw (415) and female screw (425) it is obvious.

While it is made of the shape of the cylinder and the shape is comprised of the working electrode part (470), the electrode separation large (460) and ground electrode (430) the small-sized vibrating motor (477) for the vibration is adhered in the working electrode part (470) bottom surface between the magnet (magnet) (475) in which the penetration hole (475-1) of the center is formed and the massage apparatus part (490) is installed.

It is installed between the ground electrode (430) and working electrode part (470) and the electrode separation large (460) each other severs one's connection with the ground electrode and working electrode liver. This electrode separation large (460) is included of the shape of cylindrical and while the lower part of the ground electrode (430) is combined, the penetration hole (462) is formed in inside.

The snuggly wall (not illustrated) the circuit board (451) having the battery (450) and current generation circuit are placed are formed in the opened top of the ground electrode (430). And the lower part is shut tightly. In the meantime, the ground electrode (430) has the polarity of the working electrode part (470) which will be described later, each other, the opposite polarity.

While being settled by the circuit board (451) in the opened top of the ground electrode (430) the battery (450) is fixed in the bottom plane of the ground electrode (430) with the opening/closing device. In the back side of the circuit board (451), the lead line (453) which electrically connects the battery (450) and ground electrode (430) is formed. Using the power source of the battery (450) in the circuit board (451), it has the electronic circuit (the not illustrated) driving the ion migration current (the galvanic ionic current) and small-sized vibrating motor (477).

In the meantime, the power switch (440) is connected to the electronic circuit of the circuit board (451) through the ground electrode (430) through-hole (435). According to the power switch (440) is the property of the container, it is installed at the electrode separation large (460). According to this power switch (440) is the operation of the user, the supply of the ionic current and motor driving current is switched with the working electrode part (470) in the electronic circuit in the ground electrode (430) and small-sized vibrating motor (477). The working electrode part (470) and ground electrode (430) may be formed of the synthetic resin which is coated with the metal or the conductive material so that the current flows.

In the meantime, is side installed through the circuit board (430) the circumference of the electrode separation large (460) and the display lamp (480) indicates the decided operation of the working electrode part (470). The display lamp (480) can comprise so that it is turned on if the human body is contacted with the contact unit (not illustrated) with the human detection sensor (not illustrated) separately installed at the contact unit (not illustrated).

It is provided with the power source from the battery (450) and it shares the drive of the small-sized vibrating motor (477) and iontophoresis generation and the skin absorption rate of the product is enhanced the circuit board (451) electronic circuit.

Moreover, for the more minute vibration, the supersonic vibration can be used.

As shown in FIG. 11. And this ultrasonic massage device using the magnet according to the embodiment 5 is the cosmetic container (400) adhered to the lower part of the present invention is equipped with the receiving part (410), the lid part (420) ground electrode (430), the battery (450), the circuit board (451), the power switch (440), the display lamp (480), the electrode separation large (460), the working electrode part (470), the piezoelectric element (477-1) etc.

While being included of the shape of cylinder and being composed of the working electrode part (470), the electrode separation large (460) and ground electrode (430), the piezoelectric element (477-1) for the supersonic vibration is attached in the working electrode part (470) bottom surface between the magnet (magnet) (475) in which for center, the penetration hole (475-1) is formed and the massage apparatus part (490) is installed.

The snuggly wall (not illustrated) in which circuit board (451) having the battery (450) in the opened top of the ground electrode (430) and ultrasonic generation circuit are placed is formed. In the meantime, the ground electrode (430) has the polarity of the working electrode part (470) which will be described later, each other, the opposite polarity.

While being settled by the circuit board (451) in the opened top of the ground electrode (460) the battery (450) is fixed in the bottom plane of the ground electrode (460) with opening and closing part. In the back side of the circuit board (451), the lead line (453) which electrically connects the battery (450) and ground electrode (430) is installed. Using the power source of the battery (450) in the circuit board (451), it has the ion migration current (the galvanic ionic current) and the electronic circuit (the not illustrated) generating the ultrasound current.

In the meantime, the power switch (440) is connected to the electronic circuit of the circuit board (451) through the through-hole (435) of the ground electrode (430). According to the power switch (440) is the property of the container, it is installed of the electrode separation large (460). The supply of the ionic current and ultrasound current is switched with the working electrode part (470) in which this power switch (440) is the ground electrode (430) and ultrasound piezoelectric element (477-1) in the electronic circuit according to the operation of the user are installed. The working electrode part (470) and ground electrode (430) may be formed of the synthetic resin which is coated with the metal or the conductive material so that the current flows.

In the meantime, it is side installed through the circuit board (430) the circumference of the electrode separation large (460) and the display lamp (480) indicates the decided operation of the working electrode part (470). The display lamp (480) can comprise so that it is turned on if the human body is contacted with the contact unit (not illustrated) with the human detection sensor (not illustrated) separately installed at the contact unit (not illustrated).

The circuit board (451) electronic circuit comprises the iontophoresis it is provided with the power source from the battery (450), the frequency Generator (not illustrated), generating the ultra-audible frequency and iontophoresis, and the amplifier (not illustrated), amplifying the frequency generated in the frequency Generator (not illustrated), in other words, the iontophoresis and ultra-audible frequency and controller and MCU (micro controller unit) (not illustrated) etc. authorizes the amplified iontophoresis as described above and ultra-audible frequency for the ultrasonic generation of the working electrode part (470) bottom surface and working electrode part (470) to the piezoelectric vibrator (477-1).

It is installed between the ground electrode (430) and working electrode part (470) and the electrode separation large (460) insulates the ground electrode and working electrode liver. This electrode separation large (460) is made of the cylindrical shape and the penetration hole (462) is formed in the inside so that the lower part of the ground electrode (430) is inserted. And in the inner circumference of the electrode separation large (460), the corresponding joints (461-a, 461-b) is formed as the regular length so that the working electrode part (470) which will be described later and ground electrode (430) are combined. It is formed with the transparent material and it makes the gradual rise light of the display lamp (480) transmitted to this electrode separation large (460) in the lighting of the display lamp (480) which will be described later to the outside of the separator (460). Moreover, it has it forms the through-hole (481) of the uncertain separator and it lights the display lamp.

Supplies the ultra-fine vibration to the human body in which the ultrasound piezoelectric element (477-1) is adhered in the working electrode (470) top bottom surface to the insulating adhesive (476) and which is provided with the ultra-audible frequency from the circuit board (451) and which is contacted through the working electrode part (470). It is made of the form in which the conductive material of the metallic component with high working electrode part (470) is silver, and the conductivity like gold or the copper or the synthetic resin is plated. This working electrode part (470) comprises the main body, the joint (the not illustrated), the contact unit (474), the lead line (438) etc. While the ultrasound piezoelectric element (477-1) is fixed to the bottom surface of the working electrode part (470) to the insulated adhesive (476) the insulating adhesive (476) can be the alumina (Al2O3).

Moreover, the penetrated magnet (magnet) (475) is adhered and the ultrasound piezoelectric element (477-1) adhered to the working electrode part (470) and insulating adhesive (476) can massage the ultrasound massage, ionization and magnetic field massage in single it is of course.

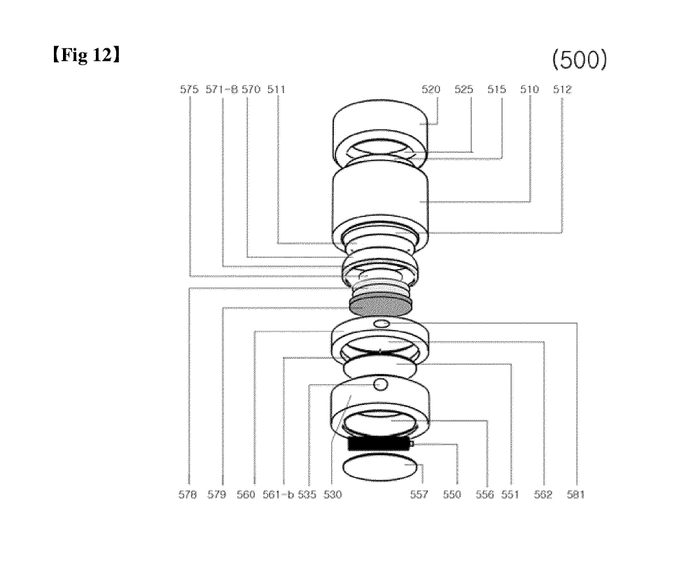

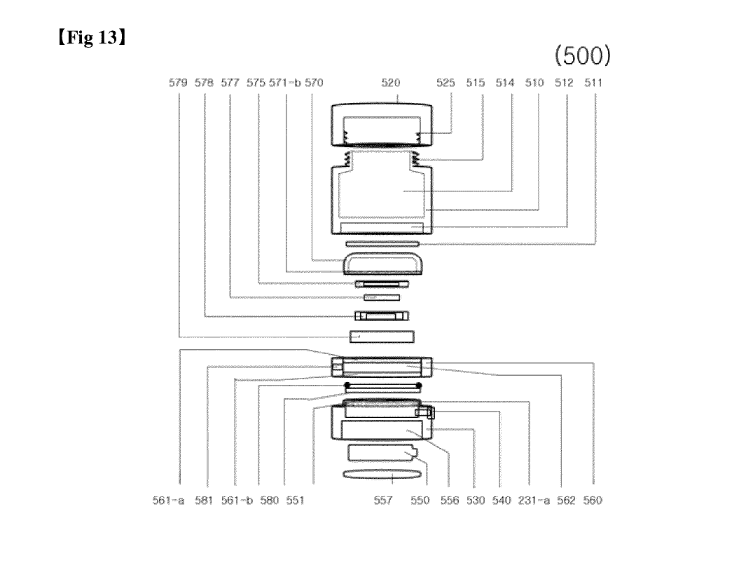

FIG. 12 is isolation showing the configuration of the cosmetics container in which cold and warmth massage apparatus using the magnet according to the working example 6 of the present invention are attached to the lower part. FIG. 13 is a side sectional view showing the configuration of the cosmetics container in which cold and warmth massage apparatus using the magnet according to the working example 6 of the present invention are attached to the lower part.

As shown in FIGS. 12 and 13. And the massage apparatus using the magnet according to the embodiment 6 is the cosmetic container (500) adhered to the lower part of the present invention is equipped with the receiving part (510), the lid part (520) ground electrode (530), the battery (550), the circuit board (551), the power switch (540), the display lamp (580), the electrode separation large (560), the working electrode part (570), the radiator (578), the heat radiation fan (579), the thermoelectric element (577) etc.

The receiving part (510) is included of the shape of cylinder and the top is opened and the lower part is shut tightly and the cradle groove (512) is left in the bottom surface and the magnetic material (511) is placed. In the inner circumference of the lower part of the lid part (520), in order to be screwed with the opened male screw (515) formed in upper part which of the receiving part (510) the female screw (525) is formed as the regular length.

While it is made of the shape of the cylinder and the shape is comprised of the working electrode part (570), the electrode separation large (560) and ground electrode (530) the thermoelectric element (577) for the generation which the coldness comes is adhered in the working electrode part (570) bottom surface between the magnet (magnet) (575) in which the penetration hole (575-1) is formed in the center and the massage apparatus part (590) is installed.

The snuggly wall (not illustrated) in which the circuit board (551) having the battery (550) and cold and warmth generation circuit are placed are formed in the opened top of the ground electrode (530). And the lower part is shut tightly. In the meantime, the ground electrode (530) has the polarity of the working electrode part (570) which will be described later, each other, the opposite polarity.

In the back side of the thermoelectric element (577), the lead line (553) which electrically connects the battery (550) and circuit board (551) while the radiator (578) and heat radiation fan (579) are installed is formed. Using the power source of the battery (550) in the circuit board (551), it has the ion migration current (the galvanic ionic current) and the electronic circuit (the not illustrated) generating cold and warmth current.

In the meantime, it is circulated with the through-hole (553) of the ground electrode (530) and the power switch (540) is connected to the electronic circuit of the circuit board (551). Moreover, according to the power switch (540) is the property of the container, it is installed at the electrode separation large (560). The supply of the ionic current and coldness on current is switched with the working electrode part (570) in which this power switch (540) is the ground electrode (530) and thermoelectric element (577) in the electronic circuit according to the operation of the user are installed. The working electrode part (570) and ground electrode (530) may be formed of the synthetic resin which is coated with the metal or the conductive material so that the current flows.

In the meantime, is side installed through the circuit board (530) the circumference of the electrode separation large (560) and the display lamp (580) indicates the decided operation of the working electrode part (570). The display lamp (580) can comprise so that it is turned on if the human body is contacted with the contact unit (not illustrated) with the human detection sensor (not illustrated) separately installed at the contact unit (not illustrated).

The circuit board (551) electronic circuit comprises the iontophoresis it is provided with the power source from the battery (550), the working electrode part (570) in the electric current supply (not illustrated) generating cold and warmth current, and the controller and MCU (micro controller unit) (not illustrated) etc. it is applied for the generation which the coldness of the working electrode part (570) bottom surface comes to the thermoelectric element (577).

It is installed between the ground electrode (530) and working electrode part (570) and the electrode separation large (560) insulates the ground electrode and working electrode liver. This electrode separation large (560) is made of the cylindrical shape and the penetration hole (562) is formed in the inside so that the lower part of the ground electrode (530) is combined. And in the inner circumference of the electrode separation large (560), the corresponding joints (561-a, 561-b) is formed as the regular length so that the working electrode part (570) which will be described later and ground electrode (530) are combined. It is formed with the transparent material and it makes the gradual rise light of the display lamp (580) transmitted to this electrode separation large (560) in the lighting of the display lamp (580) which will be described later to the outside of the separator (560).

Supplies cold and hot or the heat to the human body in which cold and warmth heat burnout person (577) are attached in the working electrode part (570) top bottom surface and in which current is provided from the circuit board (551) and which is touched through the working electrode part (570). It is included of the form in which the conductive material is plated in the metallic component with high working electrode part (470) is the silver, and the conductivity like the gold or copper or the synthetic resin. This working electrode part (570) comprises the main body, the joint (the non illustration), the contact unit (the non illustration), the lead line (553) etc.

Moreover, the penetrated magnet (magnet) (575) is attached and the thermoelectric element (577) coming the working electrode part (570) and the coldness which attaches to cold and warmth massage, the ionization and magnetic field massage in the single, it is of course.

Hereinafter, the massage apparatus using magnet according to the embodiments 1 through 6 of the present invention illustrates the combining relation of the cosmetic container adhered to lower part.

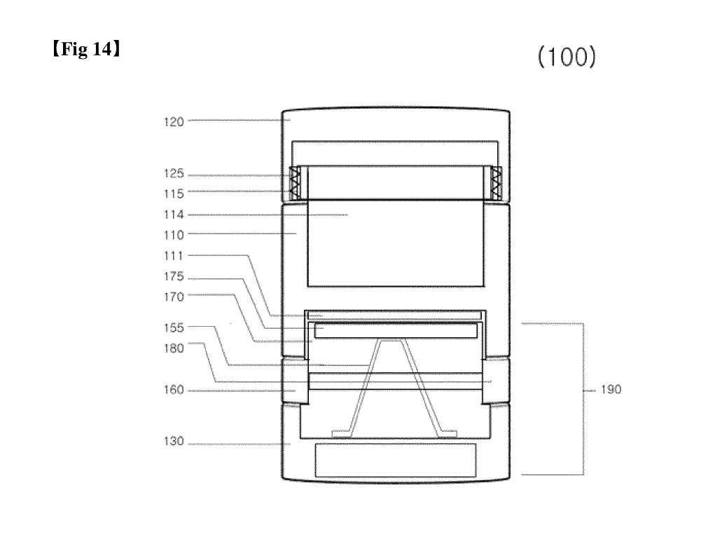

FIG. 14 is a side sectional view showing the display in which the cosmetic container adhered to the lower part of the massage apparatus using the magnet according to the embodiment 1 of the present invention is combined. And the female screw (125) formed in the lower part of the male screw (115) and the lid part (120) which is opened in order to put the aqueous solution and top is formed into the perimeter wall of the massage apparatus using FIG. 14 is the magnet of the embodiment 1 of the present invention is combined as shown in FIG. 2 in the form of the container capable of containing the receiving part (110) is the general cosmetics of the cosmetic container (100) adhered to the lower part to the screw thread. And in the sealed lower part bottom surface of the receiving part (110), the magnetic material (111) or the magnet which and it follows is adhered so that the installed magnet (magnet) (175) of the massage apparatus part (190) is adhered.

While being composed of the work nest (170), the separator (160) and ground (130), it can be detached and attached, in the massage apparatus part (190), the magnet (magnet) (175) is installed in cylindrical with the receiving part (110) lower part in the work nest (170) bottom surface. The top of the work nest (170) is shut tightly and the lower part is opened and the separator (160) is installed at the ground (130) and bond. The massage apparatus part (190) established in the receiving part (110) for the magnetic material (111) and bond but the magnet (magnet) (175) can massage the magnetic field using the magnet (magnet) (175). The joint (171-a) in which the top is shut tightly and in which the magnet (magnet) (175) of the south pole or N pole is attached to the bottom surface and in which the lower part is opened and coping with the joint (161-a) of the upper part of the separator (160) is formed and the work nest (170) uniting with the massage apparatus part (190) with the receiving part lower part is combined. And in the separator (160) is the top and lower part, joints (161-a, 161-b) are respectively formed. While in form the top in which in the ground (130), the top is opened and the lower part is shut tightly, the joint (131-a) is formed for the bottom combination part (161-b) and connection of separator, the opposite pole N pole or the south pole of the magnet (magnet) (175) attached to the work nest (170) is connected to the rear side.

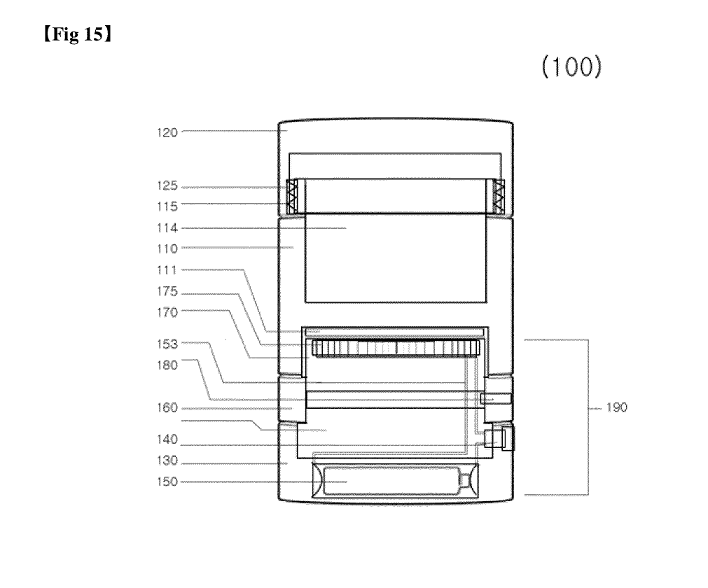

FIG. 15 is a side sectional view showing the display in which the cosmetic container adhered to the lower part of the electromagnet massage apparatus using the magnet according to the embodiment 1 of the present invention is combined. And the female screw (125) formed in the lower part of the male screw (115) and the lid part (120) which is opened in order to put the aqueous solution and top is formed into the perimeter wall of the electromagnet massage apparatus using FIG. 15 is the magnet of the embodiment 1 of the present invention as to the cosmetic container (100) adhered to the lower part, is combined as shown in FIG. 3 in the form of the container capable of containing the general cosmetics to the screw thread. And in the sealed lower part bottom surface of the receiving part (110), the magnetic material (111) or the magnet which and it follows is adhered so that the installed magnet (magnet) (175) of the massage apparatus part (190) is adhered.

The magnetic field is formed into the principles of the electromagnet and using this structure is the power source of the battery (150) it is adhered to the work nest (170) bottom surface to the structure where the coil of the metal in which the penetration hole is formed instead of the magnet of the massage apparatus part (190) is wound, it can be the magnetic force of the magnetic field to the power switch misrepresentation operation with on/off. In order to do that, while the metal unit (173) is formed in the bottom surface into the working electrode part (170-1) the coil (176) is applied about the power source from the metal unit (173) outer circumference in cold the battery (150) and the work nest (170) can form the magnetic field into the power switch misrepresentation (140) operation. It becomes the connecting part (130), moreover, the ground electrode (130-1) and the opposed polarity of the working electrode part (170-1) of the battery (150) is connected. And the electrode separation large (160) was installed between the ground electrode (130-1) and working electrode part (170-1) and it was not the ground electrode and working electrode liver connected. While the magnetic field massage apparatus part (190) in which the connection structure is applied about the power source from the battery (150) and operating the negative current (−) of the battery is connected to the working electrode (170-1) from the metal unit (173) the positive electric current (+) is connected through the power switch (140) to the ground electrode (130-1). While being installed at the through-hole (135) of the ground electrode (130) the power switch (140) switching the power source can look upon the ion transmission effect with the battery (150). Here, while expressing the lighting device of the body perception display lamp in the simple circuit configuration of the transistor or the diode and resistance and led the display lamp is set up in the electrode separation large (160-1) and it visually can confirm. Moreover, in order to the ground electrode (170) lower part be shut tightly and make the battery exchange easy the battery cradle groove (156) and battery cover (157) are installed.

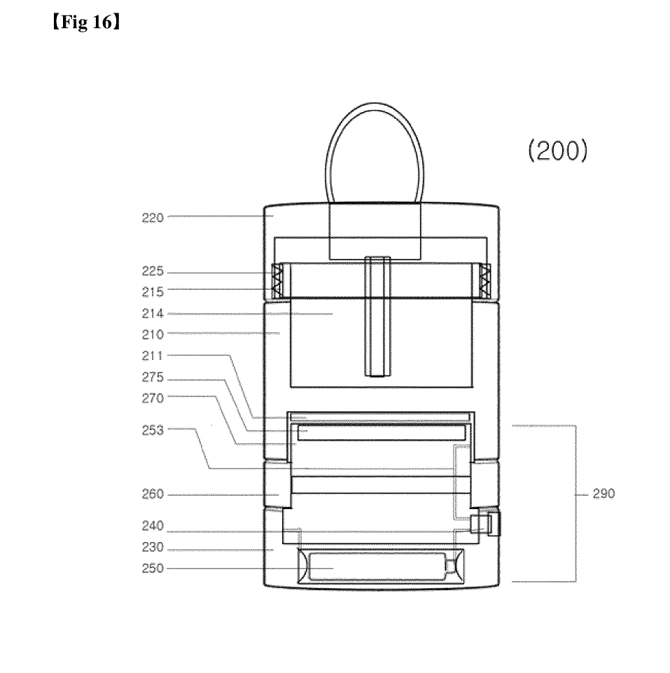

FIG. 16 is a side sectional view showing the display in which the cosmetics container in which the ionization massage apparatus using the magnet according to the working example 2 of the present invention is attached to the lower part is combined. And in as to FIG. 16 of the working example 2 of the present invention, the cosmetics container (200) in which the ion massage apparatus using magnet is attached to the lower part, the female screw (225) formed in the lower part of the male screw (215) and the lid part (220) which is opened in order to contain the aqueous solution and the top is formed into the perimeter wall is combined as shown in FIG. 4, and the FIGS. 5 through 16 as shown in FIG. 2 in the form of the container capable of containing the receiving part (210) as to the cosmetics container (200), in which in FIG. 14, the massage apparatus using magnet is attached to the lower part is the general cosmetic of the working example 1 of the present invention to the screw thread. And in the lower part bottom surface shut tightly of the receiving part (210), the magnetic material (211) or the magnet which it and follows is attached. Thus, the invention relates to user is the forward direction or the reverse direction the lid part (220) of the cosmetics container (200) in the receiving part (210). And it can stick as drop-out while revolving.

It is made of the shape of the cylinder and it draws and the massage apparatus part (290) is connected in the ground electrode (230).

While the working electrode part (270) is to the synthetic resin coated with the metal or the conductive material so that the current flows the top upper side is shut tightly and the magnet (magnet) (275) for the magnetic material (211) and attaching is installed in the shape of the cylinder in the bottom surface at the lower part of the receiving part. The lower part has housing and it is opened and the joint (271-b) is formed.

While the corresponding joint (261-a) is formed so that top link to the joint (271-1) of the working electrode part (270) lower part while the electrode separation large (260) the penetration hole (262) is formed in the center the joint (261-b) is formed in the lower part. And in the electrode separation large outer circumference, the through-hole (281) is formed in order to set up the display lamp (280).

So that the ground electrode (230) be connected to the joint (261-b) of the lower part of the electrode separation large (260) while it is opened while it consists of the synthetic resin coated with the metal or the conductive material so that the current flows having top is housing to the shape of the cylinder the corresponding joint (231-a) is formed. And the battery cradle groove (256) it makes the exchange of the battery (250) easy is formed in the lower part bottom of bottom side. And the through-hole (235) is formed so that the power switch (240) is installed in the outer circumference while the battery cover (257) is installed. Moreover, the hanging projection (the not illustrated) for being placed while the circuit board (251) having the electronic circuit in the inner structure of housing is installed is installed.

The circuit board (251) having the working electrode part (270) of the massage apparatus part (290) and ground electrode (230) and battery (250) and power switch (240) and electronic circuit is each other connected to the lead line (253) of the conductive material. The ion massage flowing in the skin which is burned earth current (the negative ion (−), and the cation (+)) is the human body if the ground electrode (230) is caught as the hand after pasting and user flows cosmetic after the power operation in the skin and task current (the cation (+), and the negative ion (−)) are touched if the working electrode part (270) is touched in the skin and helps the absorption and exhaustion of cosmetic according to polarity ((the negative ion (−), and the cation (+)) can be.

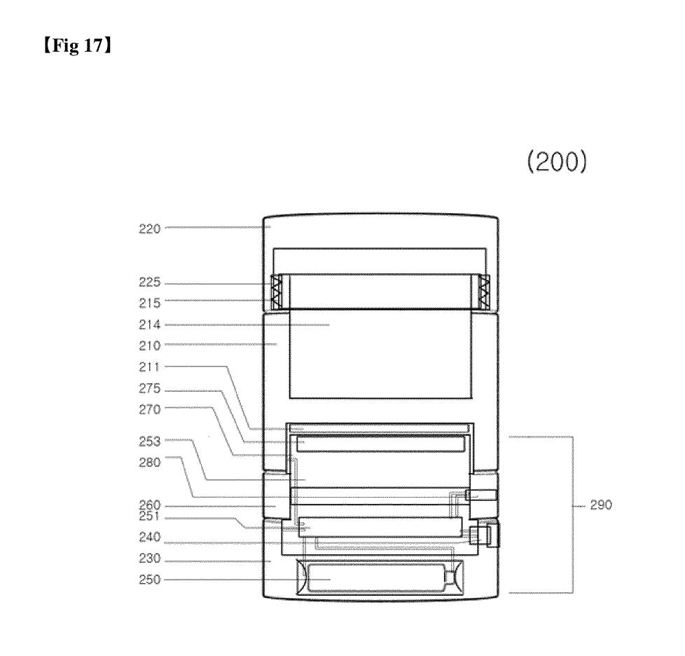

FIG. 17 is a side sectional view showing the display in which the cosmetics container in which the iontophoresis massage apparatus using the magnet according to the working example 3 of the present invention is attached to the lower part is combined. And the female screw (225) formed in the lower part of the male screw (215) and the lid part (220) which is opened in order to contain the aqueous solution and the top is formed into the perimeter wall is combined as shown in the FIG. 6, and the FIGS. 7 through 17 in the form of the container capable of containing the receiving part (210) as to the cosmetics container (200), in which in FIG. 17, the iontophoresis massage apparatus using magnet is attached to the lower part is the general cosmetic of the working example 3 of the present invention to the screw thread. And in the lower part bottom surface shut tightly of the receiving part (210), the magnetic material (211) or the magnet which it and follows is attached. Thus, the invention relates to user is the forward direction or the reverse direction the lid part (220) of the cosmetics container (200) in the receiving part (210). And it can stick as drop-out while revolving.

It is made of the shape of the cylinder and it draws and the massage apparatus part (290) is connected in the ground electrode (230).

While the working electrode part (270) is to the synthetic resin coated with the metal or the conductive material so that the current flows the top upper side is shut tightly and the magnet (magnet) (275) for the magnetic material (211) and attaching is installed in the shape of the cylinder in the bottom surface at the lower part of the receiving part. The lower part has housing and it is opened and the joint (271-b) is formed.

While the corresponding joint (261-a) is formed so that top link to the joint (271-1) of the working electrode part (270) lower part while the electrode separation large (260) the penetration hole (262) is formed in the center the joint (261-b) is formed in the lower part. And in the electrode separation large outer circumference, the through-hole (281) is formed in order to set up the display lamp (280).

So that the ground electrode (230) be connected to the joint (261-b) of the lower part of the electrode separation large (260) while it is opened while it consists of the synthetic resin coated with the metal or the conductive material so that the current flows having top is housing to the shape of the cylinder the corresponding joint (231-a) is formed. And the battery cradle groove (256) it makes the exchange of the battery (250) easy is formed in the lower part bottom of bottom side. And the through-hole (235) is formed so that the power switch (240) is installed in the outer circumference while the battery cover (257) is installed. Moreover, the hanging projection (the not illustrated) for being placed while the circuit board (251) having the MCU (micro controller unit) electronic circuit in the inner structure of housing is installed is installed.

Circuit board (251) having the working electrode part (270) of the massage apparatus part (290) and ground electrode (230) and battery (250) and power switch (240) and electronic circuit are connected to the lead line (253) of the conductive material. The iontophoresis massage flowing into the skin in which jumps on earth current (the negative ion (−), and the cation (+)) is the human body if the ground electrode (230) is by hand caught after it is right and the user flows the cosmetics after the power operation in skin and task current (the cation (+), and the negative ion (−)) are contacted if the skin contacts the working electrode part (270) in skin and helps absorption and exhaustion of the cosmetics according to polarity ((the negative ion (−), and the cation (+)) can be massaged.

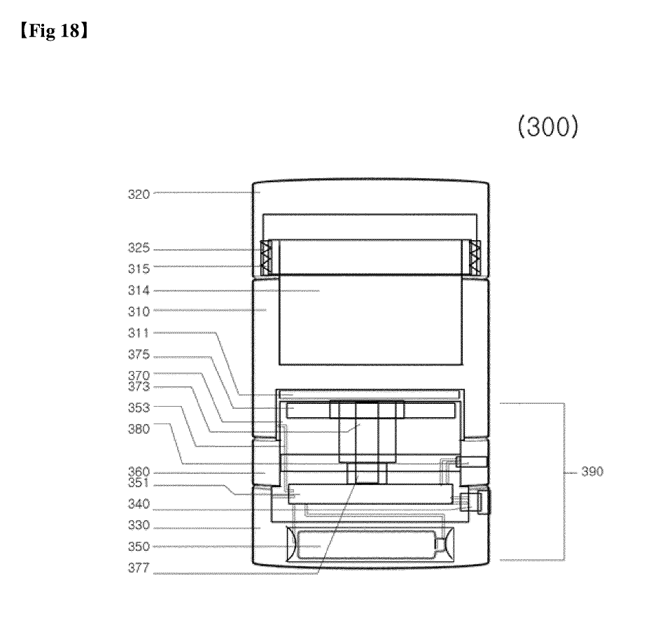

FIG. 8 is an isolation and side sectional view showing the display in which the cosmetic container adhered to the lower part of the skin laser massage apparatus using the magnet according to the embodiment 4 of the present invention is combined. And as to FIG. 8, and the skin laser massage apparatus using FIG. 18 is the magnet of the embodiment 4 of the present invention as shown in FIG. 18 is the cosmetic container (300) adhered to the lower part, the female screw (325) formed in the lower part of the male screw (315) and the lid part (320) which is opened in order to put the aqueous solution and top is formed into the perimeter wall are combined in the form of the container capable of containing the receiving part (310) is the general cosmetics to the screw thread. And in the sealed lower part bottom surface of the receiving part (310), the magnetic material (311) or the magnet which and it follows is adhered. Thus, the invention relates to the user is the regular direction or the backward direction the lid part (320) of the cosmetic container (300) in the receiving part (310). And it can attach as the drop-out while rotating.

It is made of the shape of the cylinder and it draws and the massage apparatus part (390) is connected in the connecting part (330).

The penetration hole (372-1) in the center is formed in the work nest (370) is the shape of the cylinder the top upper side. And the magnet (magnet) (375) having the penetration hole (375-1) for the magnetic material (311) at the lower part of the receiving part and attaching are installed in the bottom surface. And for the laser irradiation among the penetration hole (375-1). While the guide tube (373) is installed the lower part has housing and it is opened and the joint (371-b) is formed.

While the corresponding joint (361-a) is formed so that top link to the joint (371-1) of the working electrode part (370) lower part while the electrode separation large (360) the penetration hole (362) is formed in the center the joint (361-b) is formed in the lower part.

So that the connecting part (330) be connected to the joint (361-b) of the lower part of the electrode separation large (360) while top is open to the shape of the cylinder having housing the corresponding joint (331-a) is formed. And the battery cradle groove (356) it makes the exchange of the battery (350) easy is formed in the lower part bottom of bottom side. And the through-hole (335) is formed so that the power switch (340) is installed in the outer circumference while the battery cover (357) is installed. Moreover, the hanging projection (the not illustrated) for being placed while the circuit board (351) having the laser-irradiated region (377) in the inner structure of housing and electronic circuit are installed is installed.

Circuit board (351) having the work nest (370) of the massage apparatus part (390) and connecting part (330) and battery (350) and power switch (340) and electronic circuit are connected to the lead line (353) of the conductive material. It can massage with skin laser that increases the acne treatment through the laser irradiation of the work nest (370) or the skin regeneration power the user catches by hand the connecting part (330) after the power operation in skin after it is right.

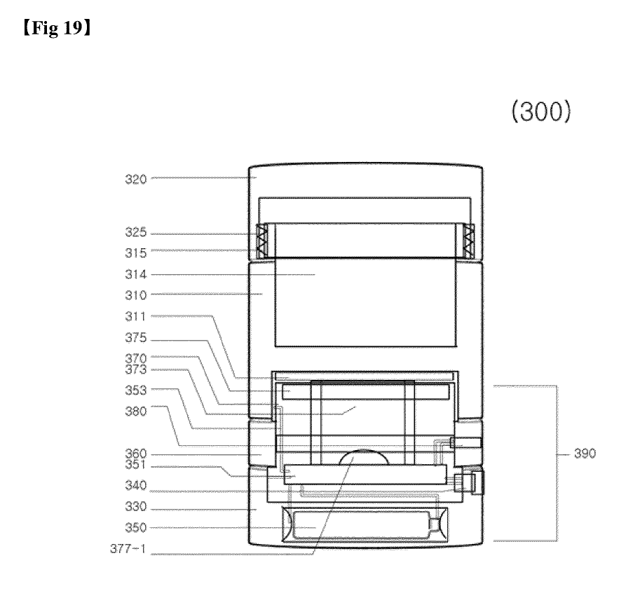

FIG. 19 is a side sectional view showing the display in which the cosmetic container adhered to the lower part of the optical irradiation massage apparatus using the magnet is combined in the embodiment 4 of the present invention. And as to FIG. 9, and the optical irradiation massage apparatus using FIG. 19 is the magnet of the embodiment 4 of the present invention as shown in FIG. 19 is the cosmetic container (300) adhered to the lower part, the female screw (325) formed in the lower part of the male screw (315) and the lid part (320) which is opened in order to put the aqueous solution and top is formed into the perimeter wall are combined in the form of the container capable of containing the receiving part (310) is the general cosmetics to the screw thread. And in the sealed lower part bottom surface of the receiving part (310), the magnetic material (311) or the magnet which and it follows is adhered. Thus, the invention relates to the user is the regular direction or the backward direction the lid part (320) of the cosmetic container (300) in the receiving part (310). And it can attach as the drop-out while rotating.

It is made of the shape of the cylinder and it draws and the massage apparatus part (390) is connected in the ground electrode (330-1).

The penetration hole (372-1) in the center is formed in the working electrode part (370-1) is the shape of the cylinder the top upper side. And the magnet (magnet) (375) having the penetration hole (375-1) for the magnetic material (311) at the lower part of the receiving part and attaching are installed in the bottom surface. And while the guide tube (373) for the optical irradiation is installed between the penetration hole (375-1) the lower part has housing and it is opened and the joint (371-b) is formed.

While the joint (361-a) which copes with order to the top be connected to the joint (371-1) of the working electrode part (370-1) lower part while in the electrode separation large (360), the penetration hole (362) is formed in center is formed, the joint (361-b) is formed in the lower part.

So that the ground electrode (330-1) be connected to the joint (361-b) of the lower part of the electrode separation large (360) while top is open to the shape of the cylinder having housing the corresponding joint (331-a) is formed. And the battery cradle groove (356) it makes the exchange of the battery (350) easy is formed in the lower part bottom of bottom side. And the through-hole (335) is formed so that the power switch (340) is installed in the outer circumference while the battery cover (357) is installed. Moreover, the hanging projection (the not illustrated) for being placed while the circuit board (351) having the illuminating unit (377-1) in the inner structure of housing and electronic circuit are installed is installed.

The circuit board (351) having the working electrode part (370-1) of the massage apparatus part (390) and ground electrode (330-1) and battery (350) and power switch (340) and electronic circuit is each other connected to the lead line (353) of the conductive material. The ion massage and the optical irradiation massage flowing in the skin which is burned earth current (the negative ion (−), and the cation (+)) is the human body if the ground electrode (330) is caught as the hand after pasting and user flows cosmetic after the power operation in the skin and task current (the cation (+), and the negative ion (−)) are touched if the working electrode part (370) is touched in the skin and helps the absorption and exhaustion of cosmetic according to polarity ((the negative ion (−), and the cation (+)) can be together. In the ionic degree joining, it can use only the optical irradiation masseur machine which uses the illuminating unit (377) if the connected lead line (353) of the working electrode part (370) is eliminated.

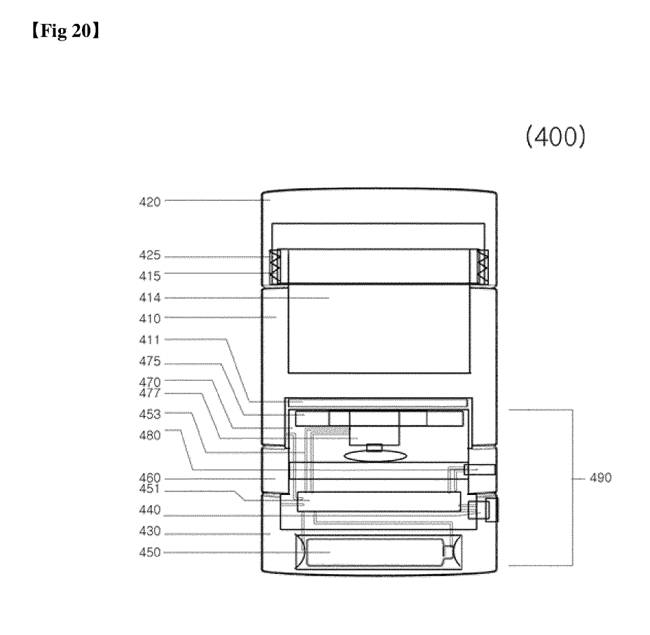

FIG. 20 is a side sectional view showing the display in which the cosmetic container adhered to the lower part of the vibration massage apparatus using the magnet is combined in the embodiment 5 of the present invention. And as to FIG. 10, and the vibration massage apparatus using FIG. 20 is the magnet of the embodiment 5 of the present invention as shown in FIG. 20 is the cosmetic container (400) adhered to the lower part, the female screw (425) formed in the lower part of the male screw (415) and the lid part (420) which is opened in order to put the aqueous solution and top is formed into the perimeter wall are combined in the form of the container capable of containing the receiving part (410) is the general cosmetics to the screw thread. And in the sealed lower part bottom surface of the receiving part (410), the magnetic material (411) or the magnet which and it follows is adhered. Thus, the invention relates to the user is the regular direction or the backward direction the lid part (420) of the cosmetic container (400) in the receiving part (410). And it can attach as the drop-out while rotating.

It is made of the shape of the cylinder and it draws and the massage apparatus part (490) is connected in the ground electrode (430).

The magnet (magnet) (475) having the penetration hole (475-1) for the magnetic material (411) at the lower part of the receiving part and attaching are installed in the bottom surface while the working electrode part (470) is to the synthetic resin coated with the metal or the conductive material so that the current flows the top upper side is shut tightly with the shape of the cylinder. And while the miniature vibration motto (477) is installed at the micro-vibration between the penetration hole (475-1) the lower part has housing and it is opened and the joint (471-b) is formed.

While the corresponding joint (461-a) is formed so that top link to the joint (471-1) of the working electrode part (470) lower part while the electrode separation large (460) the penetration hole (462) is formed in the center the joint (461-b) is formed in the lower part. And in the electrode separation large outer circumference, the through-hole (481) is formed in order to set up the display lamp (480).

The joint (431-a) which copes with order to the ground electrode (430) be connected to the joint (461-b) of the lower part of the electrode separation large (460) while being opened having the top is the housing to the shape of cylinder while being to the synthetic resin coated to metal or the conductive material so that current flow is formed. And in the lower part bottom of bottom side, in order to easy the exchange of the battery (450), the through-hole (435) is formed so that the power switch (440) be installed in the outer circumference while the battery cover (457) is installed while the battery cradle groove (456) is formed. Moreover, the jaw (the non illustration) for placing while the circuit board (451) having the electronic circuit in the inner structure of housing is installed is installed.

Circuit board (451) having the working electrode part (470) of the massage apparatus part (490) and ground electrode (430) and battery (450) and power switch (440) and electronic circuit are connected to the lead line (453) of the conductive material. The tremolo massage knocking at skin with the iontophoresis massage flowing into the skin in which jumps on earth current (the negative ion (−), and the cation (+)) is the human body if the ground electrode (430) is by hand caught after it is right and the user flows the cosmetics after the power operation in skin and task current (the cation (+), and the negative ion (−)) are contacted if the skin contacts the working electrode part (470) in skin and helps absorption and exhaustion of the cosmetics according to polarity ((the negative ion (−), and the cation (+)) can be massaged. It can use only cold and warmth masseur machine using the small-sized vibrating motor (477) the connected lead line (453) of the working electrode part (470) is removed of the iontophoresis circuit.

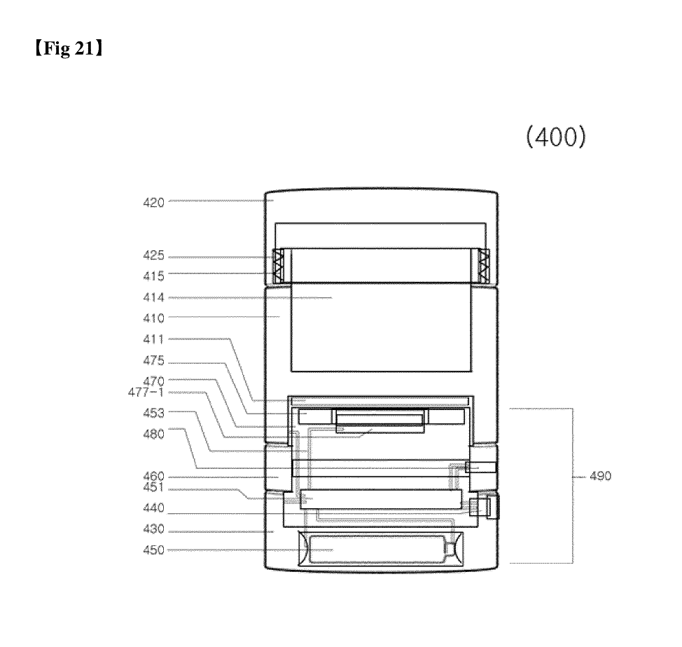

FIG. 21 is a side sectional view showing the display in which the cosmetic container adhered to the lower part of the ultrasonic massage device using the magnet is combined in the embodiment 5 of the present invention. And as to FIG. 11, and the vibration massage apparatus using FIG. 21 is the magnet of the embodiment 5 of the present invention as shown in FIG. 21 is the cosmetic container (400) adhered to the lower part, the female screw (425) formed in the lower part of the male screw (415) and the lid part (420) which is opened in order to put the aqueous solution and top is formed into the perimeter wall are combined in the form of the container capable of containing the receiving part (410) is the general cosmetics to the screw thread. And in the sealed lower part bottom surface of the receiving part (410), the magnetic material (411) or the magnet which and it follows is adhered. Thus, the invention relates to the user is the regular direction or the backward direction the lid part (420) of the cosmetic container (400) in the receiving part (410). And it can attach as the drop-out while rotating.

It is included of the shape of cylinder and it ***s the working electrode part (470) and electrode separation large (460) and the massage apparatus part (490) is connected to the ground electrode (430).

The magnet (magnet) (475) having the penetration hole (475-1) for the magnetic material (411) at the lower part of the receiving part and attaching are installed in the bottom surface while the working electrode part (470) is to the synthetic resin coated with the metal or the conductive material so that the current flows the top upper side is shut tightly with the shape of the cylinder. And while it is between the penetration hole (475-1) with ultrasonic generation and the ultrasound piezoelectric element (477) is installed the lower part has housing and it is opened and the joint (471-b) is formed. Supplies the ultra-fine vibration to the human body in which the ultrasonic oscillator (477) is adhered in the working electrode part (470) top bottom surface to the insulating adhesive (476) and which is provided with the ultra-audible frequency from the circuit board (451) and which is contacted through the working electrode part (470). While the piezoelectric vibrator (477) is fixed to the bottom surface of the working electrode part (470) to the insulated adhesive (476) the insulating adhesive (476) can be the alumina (Al2O3).

While the corresponding joint (461-a) is formed so that top link to the joint (471-1) of the working electrode part (470) lower part while the electrode separation large (460) the penetration hole (462) is formed in the center the joint (461-b) is formed in the lower part. And in the electrode separation large outer circumference, the through-hole (481) is formed in order to set up the display lamp (480).

The joint (431-a) which copes with order to the ground electrode (430) be connected to the joint (461-b) of the lower part of the electrode separation large (460) while being opened having the top is the housing to the shape of cylinder while being to the synthetic resin coated to metal or the conductive material so that current flow is formed. And in the lower part bottom of bottom side, in order to easy the exchange of the battery (450), the through-hole (435) is formed so that the power switch (440) be installed in the outer circumference while the battery cover (457) is installed while the battery cradle groove (456) is formed. Moreover, the jaw (the non illustration) for placing while the circuit board (451) having the MCU (micro controller unit) electronic circuit for the ultrasonic wave in the inner structure of housing and iontophoresis generation is installed is installed.

Circuit board (451) having the working electrode part (470) of the massage apparatus part (490) and ground electrode (430) and battery (450) and power switch (440) and electronic circuit are connected to the lead line (453) of the conductive material. At the same time, the iontophoresis massage and the supersonic vibration massage flowing into the skin in which jumps on earth current (the negative ion (−), and the cation (+)) is the human body if the ground electrode (430) is by hand caught after it is right and the user flows the cosmetics after the power operation in skin and task current (the cation (+), and the negative ion (−)) are contacted if the skin contacts the working electrode part (470) in skin and helps absorption and exhaustion of the cosmetics according to polarity ((the negative ion (−), and the cation (+)) can be massaged. It can use only the supersonic massage unit using the piezoelectric element (477-1) the connected lead line (453) of the working electrode part (470) is removed of the iontophoresis circuit.

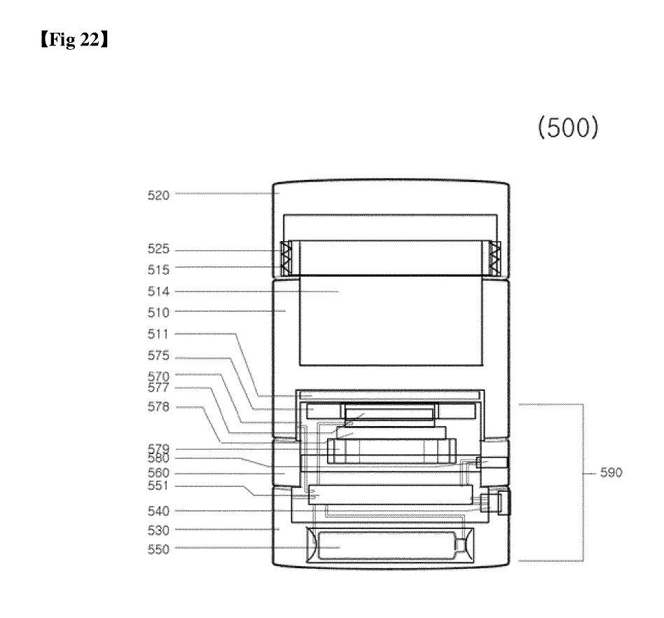

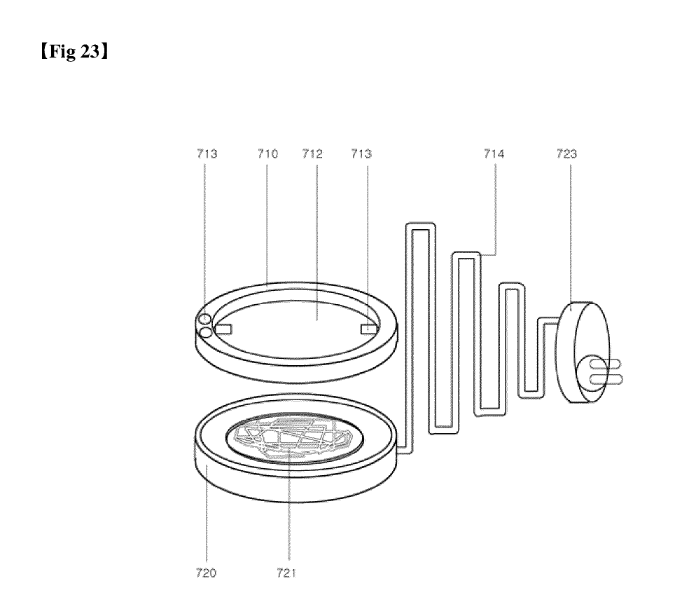

FIG. 22 is a side sectional view showing the display in which the cosmetic container adhered to the lower part of cold and warmth massage apparatus using the magnet are combined in the embodiment 6 of the present invention. And as to FIG. 12, FIG. 13, and cold and warmth massage apparatus using FIG. 22 is the magnet of the embodiment 6 of the present invention as shown in FIG. 22 is the cosmetic container (500) adhered to the lower part, the female screw (525) formed in the lower part of the male screw (515) and the lid part (520) which is opened in order to put the aqueous solution and top is formed into the perimeter wall are combined in the form of the container capable of containing the receiving part (510) is the general cosmetics to the screw thread. And in the sealed lower part bottom surface of the receiving part (510), the magnetic material (511) or the magnet which and it follows is adhered. Thus, the invention relates to the user is the regular direction or the backward direction the lid part (520) of the cosmetic container (500) in the receiving part (510). And it can attach as the drop-out while rotating.

It is made of the shape of the cylinder and it draws and the massage apparatus part (590) is connected in the ground electrode (530).

While the magnet (magnet) (575) having the penetration hole (575-1) for the magnetic material (511) at the lower part of the receiving part in the bottom surface and attach while the working electrode part (570) is to the synthetic resin coated to metal or the conductive material so that current flow, the top upper side is shut tightly with the shape of cylinder is installed and it does for the sake of cold and hot or the heat generation and cold and warmth thermoelectric element (577) are installed between the penetration hole (575-1), the lower part has the housing and it is opened and the joint (571-b) is formed.

While the joint (561-a) which copes with order to the top be connected to the joint (571-1) of the working electrode part (570) lower part while in the electrode separation large (560), the penetration hole (562) is formed in center is formed, the joint (561-b) is formed in the lower part. And in the electrode separation large outer circumference, the through-hole (581) is formed in order to establish the display lamp (580).

So that the ground electrode (530) be connected to the joint (561-b) of the lower part of the electrode separation large (560) while it is opened while it consists of the synthetic resin coated with the metal or the conductive material so that the current flows having top is housing to the shape of the cylinder the corresponding joint (531-a) is formed. And the battery cradle groove (556) it makes the exchange of the battery (550) easy is formed in the lower part bottom of bottom side. And the through-hole (535) is formed so that the power switch (540) is installed in the outer circumference while the battery cover (557) is installed. Moreover, the hanging projection (the not illustrated) for being placed while the circuit board (551) having the MCU (micro controller unit) electronic circuit for cold and hot in the inner structure of housing or heat and iontophoresis generation are installed is installed.

Circuit board (551) having the working electrode part (570) of the massage apparatus part (590) and ground electrode (530) and battery (550) and power switch (540) and electronic circuit are connected to the lead line (553) of the conductive material. It can massage with the iontophoresis massage flowing into the skin in which jumps on earth current (the negative ion (−), and the cation (+)) is the human body if the ground electrode (530) is by hand caught after it is right and the user flows the cosmetics after the power operation in skin and task current (the cation (+), and the negative ion (−)) are contacted if the skin contacts the working electrode part (570) in skin and helps absorption and exhaustion of the cosmetics according to polarity ((the negative ion (−), and the cation (+)) with cold and warmth heat. It can use only cold and warmth masseur machine using the thermoelectric element (577) the connected lead line (553) of the working electrode part is removed of the iontophoresis circuit.

23 is the magnet followed of the present invention may be referred to the side sectional view showing the structure of the independent battery charger (700) for the charge of the masseuse of the cosmetic container adhered to the massage apparatus used is the lower part. And the led display lamp (713) which informs the charge amount while the storing groove (712) in which the battery charger top main body (710) is the charger terminal apparatus (711) according to the massage apparatus part (790) infrastructure for the charge of the masseuse is installed is formed is installed. And the battery charger lower part main body (720) is the lower part main body (750) in which the cable (714) the battery charger (700) inside is provided with the power source in one side while being combined with top to the corresponding coupling structure while the floor is while the circuit board (721) for the charge is installed and for delivering to the charger terminal apparatus (713) and circuit board (721) of the top main body (710) is mounted onto this lure. And due to the cable (714) and the adapter (723) is connected.

The or more, and the invention were explained by the restricted configuration and drawing. But the technical mapping of the present invention is like that the or more, and the invention are not restricted. And the various corrections and transformation execution will be possible in the technical field in which the invention belongs with a person skilled in the art in the equivalent range of the patent claim which is written to do with the technical mapping of the present invention.

### INDUSTRIAL APPLICABILITY

Is mainly used in skin care salons and medical gineun Martha common professional skin, skin disease, according to the efficacy of the product and massage device in accordance with the characteristics used professionally. And manage the equipment is expensive and can not carry. Existing independent home mini massagers do not know about cosmetics and good, but if you use any of the massager for any cosmetic written to suit the characteristics of Cosmetic use only one kind of equipment is not being used to maximize the effectiveness of the product did not. Also to be used according to the characteristics of the product you want to use multiple small massagers discomfort was kept to type also difficult. Massager according to the present invention attached as additional structure which is attached to the housing unit and on the bottom surface of the lower housing unit that combines massage device fit massager, combined with the characteristics of the product, Medical Hospital of professional skin care and skin care to cosmetic containers customers separation professional skin care products can fit massagers attached.

For example, Vitamin C line of ultrasound features of Massa confident with Iontophoresis additional function of massage devices attached Collagen and Placenta line of cosmetic containers, cosmetic containers, cosmetic product for the treatment of acne Do with the effect of two or more limbs with laser cosmetic containers, cosmetic containers with additional massage function using LED illumination, such as cosmetics lines fullerene combined characteristics of the suit and can be sold by attaching cosmetics containers of massage device which is attached to the multi-function massage and courage to mount the advantage of being easy to carry. Efficacy of the magnet and the newly proposed to improve the flow of blood with skin care effects have an impact on the iron contained in the blood and can basically And essence ampoules contained in the product or the dropper cosmetic containers in the form of massage device for cosmetic containers installed on the bottom of the housing unit can be installed.

Here, the cosmetics container in which the massage apparatus using magnet is attached to the lower part of the present invention is equipped with the ground electrode who the massage apparatus part combined with the lower part of the receiving part provides the positive electric current (+) or the negative current (−) from battery, and the working electrode part supplying to the human body which the negative current (−) of the ground electrode and the other polarity or the positive electric current (+) is provided from battery in order to be insulated with the ground electrode while the electrode separation part is installed and touched.

In the meantime, the cosmetic container adhered to the massage apparatus used is the lower part the magnet of the present invention can further include the working electrode and ground electrode the respective negative current (−) or the circuit board which supplies the positive electric current (+) and the display lamp which is installed in the circuit board and indicates the decided operation of the working electrode it has the battery and electronic circuit it is installed in the electrode separation embolium opened in the ground electrode insulates the ground electrode and working electrode liver it is installed between the power switch: ground electrode which switches the current supply to the working electrode part in the electronic circuit it is installed in the outer circumference of the electrode separation part or the ground electrode and working electrode.

In the meantime, the outside was made of the transparent material so that the light of the display lamp was transmitted to the outside or the electrode separation part of the present invention lit the display lamp through the through-hole of the uncertain electrode separation part. Moreover, it has with the number drawing is turned on with bay when connecting the human detection sensor to the display lamp and contacting to the skin in which the user catches by hand the ground electrode and which the user calls.

In the meantime, while it can be detached and attached, the lower part of the working electrode part is combined on the top of the electrode separation part of the present invention, the penetration hole is formed.

In the meantime, the electronic circuit of the present invention is equipped with the electric current supply which generates the negative current (−) and positive electric current (+) it is provided with the power source from the battery and the polar conversion part who mutually converts the polarity of the positive electric current (+) and respective applied negative current (−) to the working electrode and ground electrode.

In the meantime, it is installed in the outer circumference of the bottom surface of the negative current generated in the voltaic occurring part and the amplifier:

controller: sanctioning the amplified negative current (−) as described above and positive electric current (+) to the respective working electrode and ground electrode and ground electrode or the electrode separation part and amplifies the positive electric current the electronic circuit of the present invention more includes the polarity changeover switch in which user can control the polar conversion part.

In the meantime, in order to the power supply unit of the present invention battery to the secondary battery and it charges the charging apparatus which is combined with the massage apparatus part attached at the lower part of the cosmetics container independenting can be established.

In the meantime, in the lower part of massage apparatus part and receiving part of the present invention, it is able to detach and attach in each other the magnetic material and magnet are installed. Moreover, it can be installed in the corresponding coupling structure in the coupling structure of the lock shape of the concavo-convex or the female screw and male screw.

In the meantime, the massage apparatus using the magnet is the cosmetic container adhered to the lower part of the present invention is equipped with the power operation switch which the illuminating unit irradiating light is installed at the inside while the massage apparatus part combining with the lower part of the receiving part the penetration hole is formed in the work nest and is provided with the current from the electronic circuit and switches through the led or the laser unit to the work nest in the connecting part or the electrode separation large outer circumference while irradiating light.

In the meantime, the massage apparatus using the magnet is the cosmetic container adhered to the lower part of the present invention is equipped with the power operation switch which the massage apparatus part combining with the lower part of the receiving part switches in the ground electrode or the electrode separation large outer circumference while being provided with the ultra-audible frequency from the electronic circuit and generating ultrasound through the piezoelectric vibrator of the working electrode part.

In the meantime, the cosmetics container in which the massage apparatus using magnet is attached to the lower part of the present invention is equipped with the working electrode part in which the flight board is the massage apparatus part combined with the lower part of the receiving part provides cold and warmth frequency from the electronic circuit and ground electrode or the power operation switch switching in the electrode separation large outer circumference the coldness or the heat is occurred through cold and warmth heat burnout person.

In the meantime, the massager of the present invention attached Cosmetic containers cosmetics containers and detachable massaging of the skin contact surface electrode grounded in plasma, plasma is generated by a cold or hot facial lymphatic flow nicely and inflammation and treatment effects.

1. The cosmetics container in which the massage apparatus using magnet is attached to the lower part wherein as the container having the masseur machine, the massage apparatus part is had in order to be combined in the receiving part having the accommodation space: lid part: who can be detached and attached, is combined in the opened top of the receiving part of the material which the magnetic material is attached to the lower part bottom surface the top is opened and lower part of the receiving part by it can be detached and attached, using magnet and massage the skin.

2. As to the massage apparatus part as to claim 1.
Working electrode part in which the magnet it combines with the receiving part lower part is installed: massage apparatus using the magnet which has the ground electrode provided with the current from the electronic circuit while being combined at the lower part of the electrode separation large: electrode separation part which insulates two electrode livers while combining with the working electrode loader and ground electrode top is the cosmetic container adhered to lower part.

3. As for claim 2, the massage apparatus using the magnet is the cosmetic container adhered to the lower part which further comprises the display lamp which is installed and has the battery and electronic circuit and is installed in the working electrode part and ground electrode in the respective negative current (−) or the circuit board which supplies the positive electric current (+): ground electrode and the electrode separation large: circuit board which is installed between the working electrode part and insulates the ground electrode and working electrode liver and indicates the decided operation of the working electrode part of the top of the power switch: ground electrode which switches the current supply to the working electrode part in the electronic circuit it is installed in the outer circumference of the ground electrode lower part or the separator.

4. As for claim 3, the cosmetic container adhered to the massage apparatus is the lower part that uses the magnet, cosmetic container adhered to the massage apparatus is the lower part comprising: the electric current supply: who generates the negative current (−) and positive electric current (+) it is provided with the power source from the battery the working electrode part and the polar conversion part who mutually converts the polarity of the positive electric current and respective negative current to the ground electrode.

5. As for claim 4, the massage apparatus using the magnet is the cosmetic container adhered to the lower part, wherein the electronic circuit further comprises the polarity changeover switch which is installed in the outer circumference of the bottom surface of the amplifier: controller: authorizing the amplified negative current (−) as described above and positive electric current (+) to the respective working electrode part and ground electrode and ground electrode or the electrode separation part and amplifies the negative current (−) generated in the electric current supply and positive electric current (+) in which the user can control the polar conversion part.

6. The cosmetics container in which the massage apparatus using magnet is attached to the lower part, wherein the massage apparatus part as to the first claim the ground electrode having the voltaic occurring part: who the power source is provided from battery while being combined at the lower part of the electrode separation large: electrode separation part in which the display lamp is installed while being combined with the working electrode lifter working electrode loader in which the small-sized vibrating motor is installed in the magnet and the penetration hole having the center perforation in the bottom surface the upper side is shut tightly and occurs the negative current (−) and positive electric current (+) and switching arrangement and provides current from the electronic circuit is had.

7. The cosmetics container in which the massage apparatus using magnet is attached to the lower part, wherein the massage apparatus part as to the first claim the ground electrode having the voltaic occurring part: who the power source is provided from battery while being combined at the lower part of the electrode separation large: electrode separation part in which the display lamp is installed while being combined with the working electrode lifter working electrode loader in which the ultrasonic wave piezoelectric element is installed in the magnet and the penetration hole having the penetration hole in the bottom surface the upper side is shut tightly and occurs the negative current (−) and positive electric current (+) and switching arrangement and provides the ultrasonic wave and ionic current from the MCU (Micro Controller Unit) electronic circuit is had.

8. The massage apparatus using the magnet is the cosmetic container adhered to the lower part, wherein the massage apparatus part as to claim 1 has the ground electrode who is provided with the power source from the battery while being combined in the magnet and the penetration hole having the penetration hole in the bottom surface the upper side is shut tightly at the lower part of the electrode separation part in which the working electrode part: display lamp which consists to the metal while the thermoelectric element is installed it combines with the working electrode loader is installed: electrode separation part and has the switching arrangement and is provided with cold and warmth current from the MCU (Micro Controller Unit) electronic circuit.

9. The cosmetics container in which the massage apparatus using magnet is attached to the lower part, wherein the massage apparatus part as to the first claim the power source is provided from battery and the ground electrode having the switching arrangement and provides current from the electronic circuit for the laser irradiation is had while being combined in the magnet and the penetration hole having the penetration hole in the bottom surface the center of the upper side is formed for the laser irradiation the penetration pestle at the lower part of the working electrode part: electrode separation large: electrode separation part combined with the working electrode loader the laser irradiation guide tube is installed in interval.

10. The massage apparatus using the magnet is the cosmetic container adhered to the lower part, wherein the massage apparatus part as to claim 1 has the ground electrode who is provided with the power source from the battery while being combined in the magnet and the penetration hole having the penetration hole in the bottom surface the glass cover in which the center of the upper side is built up for the led investigation with the penetration pestle and preventing the penetration hole is installed at the lower part of the working electrode part in which the guide tube for the optical irradiation in the interval is installed: the electrode separation part in which the display lamp it combines with the working electrode loader is installed: electrode separation part and has the switching arrangement and is provided with the current from the electronic circuit for the optical irradiation.

11. The massage apparatus using the magnet is the cosmetic container adhered to the lower part, wherein the massage apparatus part as to claim 1 has the electric current supply: who generates the butterflies the electrode separation part in which the installed working electrode part: display lamp in which the coil is rolled it combines with the working electrode loader is installed: electrode separation part is provided with the power source from the battery it is combined in the metal having the penetration hole in the bottom surface the upper side is shut tightly at the lower part of the electrode separation part in which the installed working electrode part: display lamp in which the coil is rolled it combines with the working electrode loader is installed: electrode separation part and the ground electrode provided with the current to the switching arrangement.

12. As for claim 2, the massage apparatus using the magnet is the cosmetic container adhered to the lower part, wherein the electrode separation part comprises the transparent material the light of the display lamp is transmitted to the outside.

13. As for claim 2, opening and closing device having the ground electrode is the battery cradle groove and battery cover for the battery exchange in the lower bottom side the massage apparatus using the formed magnet is the cosmetic container adhered to lower part.

14. The lower part called the ground electrode as to the thirteenth claim the massage apparatus using the magnet in which the groove or the hanging projection is formed in order not to slide down when catching by hand is the cosmetic container adhered to lower part.

15. The massage apparatus using the magnet is the cosmetic container adhered to the lower part, wherein the adapter which permits power while being combined in the lower part which becomes the floor the connection jack for being applied about the power source from the outside is installed: connection jack is equipped in the circuit board the circuit board for the charge is installed while the cradle groove in which the charger terminal apparatus according to the massage apparatus part infrastructure as to the battery charger for the charge of the secondary battery permitting the power source of the cosmetic container is installed has the corresponding coupling structure with the top in which the led display lamp which informs the charge amount while being formed is installed: top.
